(19) United States
(12) Patent Application Publication
(10) Pub. No.: US 2022/0133139 A1
(43) Pub. Date: May 5, 2022

(54) SYSTEM AND METHOD FOR SURGERY GUIDANCE

(71) Applicant: VANDERBILT UNIVERSITY, Nashville, TN (US)

(72) Inventors: Anita Mahadevan-Jansen, Nashville, TN (US); Giju Thomas, Nashville, TN (US); Kamran Idrees, Nashville, TN (US); Carmen Solorzano, Nashville, TN (US); Parker Willmon, Nashville, TN (US); Colleen Kiernan, Nashville, TN (US)

(21) Appl. No.: 17/515,748

(22) Filed: Nov. 1, 2021

Related U.S. Application Data
(60) Provisional application No. 63/108,756, filed on Nov. 2, 2020.
(60) Provisional application No. 63/128,306, filed on Dec. 21, 2020.

(51) Int. Cl. A61B 1/06; A61B 1/07; A61B 1/04; A61B 1/00

(52) U.S. Cl. CPC A61B 1/0638; A61B 1/0646; A61B 1/0684; A61B 1/0005; A61B 1/07; A61B 1/042; A61B 1/063

(57) ABSTRACT

The invention discloses systems and methods for providing anatomical guidance in surgery. The system includes a light source configured to emit light for illuminating tissues of a target region; means for obtaining autofluorescence signals emitted from the illuminated tissues in response to the illumination; and a controller configured to process the obtained autofluorescence signal to identify target tissues.

### CROSS-REFERENCE TO RELATED PATENT APPLICATIONS

This application claims priority to and the benefit of U.S. Provisional Patent Application Serial Nos. 63/108,756, filed Nov. 2, 2020, and 63/128,306, filed Dec. 21, 2020, which are incorporated herein by reference in their entireties.

### STATEMENT AS TO RIGHTS UNDER FEDERALLY-SPONSORED RESEARCH

This invention was made with government support under Contract No. 1R01CA212147-01A1 awarded by the National Institutes of Health (NIH). The government has certain rights in the invention.

### FIELD OF THE INVENTION

The invention relates generally to optical assessments of bio-objects, and more particularly, to systems and methods of using near-infrared (NIR) autofluorescence for surgery guidance, and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Islet cells in the pancreas play a vital role in regulating blood glucose levels in the human body. Various factors can permanently damage pancreatic islet cells, including viral infections, environmental toxins, traumatic organ injuries, drug toxicity, pancreatic tumors, chronic pancreatitis and metabolic disorders, such as Type-1 diabetes that affects 1.35 million people in the United States. Long-term damage to pancreatic islets eventually leads to the pancreas being unable to produce adequate insulin. Affected individuals would then be burdened with the disability of needing life-long administration of insulin on a daily basis to just maintain normal blood glucose levels. Islet cell transplantation has been shown to be an effective method of treating insulin insufficiency and making patients insulin-independent. The process includes loosening of islets from a ‘donor’ pancreas using an enzymatic solution and separating these islets via a density gradient, often a centrifuge, following which the harvested islet cells are transplanted in the recipient by injecting these cells into the portal vein of the liver. However, the current techniques associated with extracting pancreatic islets are cumbersome and plagued with several limitations. Prolonged donor tissue incubation in the enzymatic solution could lead to decreased islet viability. Subsequently, shear stress placed on the islets during the density-gradient centrifugation can damage the extracted islet cells, rendering them nonviable. Various methods of islet cell extraction have been tested in an effort to improve the yields, but the extraction rates typically stay under 50%. To enhance the yield of islet cell extraction, it therefore becomes very crucial to (i) aid the surgeon to quickly select tissue regions dense with islet cells in the fresh donor pancreas for islet harvesting and (ii) design a modality that rapidly distinguish islets from the non-islet cells in the pancreatic tissue regions selected by the surgeon. These strategies are key to ensuring maximum yield and prolonged viability of the donor islets for a successful transplantation.

Therefore, a heretofore unaddressed need exists in the art to address the aforementioned deficiencies and inadequacies.

### SUMMARY OF THE INVENTION

In one aspect, the invention relates to a system for providing anatomical guidance in a surgery. The system comprises a light source configured to emit light for illuminating tissues of a target region; means for obtaining autofluorescence signals emitted from the illuminated tissues in response to the illumination; and a controller configured to process the obtained autofluorescence signals to identify target tissues. The target region comprises an adrenal gland, a pancreas gland, a thyroid gland, a parathyroid gland, small bowel, or any other organs. In one embodiment, the surgery is an endocrine surgery. In one embodiment, the identifying the target tissues comprises differentiating diseased tissues from healthy tissues within one organ, or differentiating one organ from other tissues or organs.

In one embodiment, the system further comprises a display for displaying images corresponding to the processed autofluorescence signals for surgical guidance.

In one embodiment, the light source comprises a laser or an LED configured to emit the light having a wavelength in a near-infrared (NIR) range of about 600-1500 nm.

In one embodiment, the means for obtaining the autofluorescence signals comprises a light delivery device optically coupled with the light source and configured to deliver the light from the light source to the tissues of the target region; and a detector configured to collect the autofluorescence signals emitted from the illuminated tissues.

In one embodiment, the light delivery device comprises one or more light guides, or one or more optical fibers.

In one embodiment, the light delivery device comprises a wavelength appropriate notch filter and a double concave lens for beam divergence.

In one embodiment, the light delivery device comprises means for focusing from the light source to the tissues of the target region.

In one embodiment, the means for obtaining the autofluorescence signal further comprises at least one lens positioned between the tissues and the detector in an optical path along which the autofluorescence signals emitted from the illuminated tissues travel, and utilized to receive the autofluorescence signals emitted from the tissues and focus them on a desired spot on the detector.

In one embodiment, the means for obtaining the autofluorescence signal further comprises at least one filter positioned between the tissues and the at least one lens in the optical path, and utilized to block unwanted wavelengths, ambient light and/or reflected light from the illuminated tissues to ensure the autofluorescence signals to be detected.

In one embodiment, the detector comprises at least one camera, and/or a spectrometer.

In one embodiment, the at least one camera comprises at least one charge-coupled device (CCD) camera, at least one complementary metal oxide semiconductor (CMOS) camera, at least one photosensor array, or a combination thereof.

In one embodiment, the controller comprises a center processing unit (CPU) or a graphics processing unit (GPU).

In another aspect, the invention relates to a system for providing anatomical guidance in surgery. The system comprises a light source configured to emit light; an optical means configured to deliver the emitted light to tissues of a target region in a living subject to illuminate the tissues, and to collect light of autofluorescence signals emitted from the tissues in response to the illumination; a detector configured to obtain the collected autofluorescence signals; and a controller in communication with the detector and configured to process the obtained autofluorescence signals to identify target tissues.

In one embodiment, the light source comprises a laser or an LED configured to emit the light having a wavelength in an NIR range of about 600-1500 nm.

In one embodiment, the optical probe comprises an excitation fiber configured to deliver the light emitted from the light source to the tissues, and a plurality of collection fibers aligned circularly around the excitation fiber configured to collect the autofluorescence signals emitted from the tissues in response to the illumination.

In one embodiment, the optical means further comprises an inline filter for preventing intrinsic autofluorescence from the optical fiber itself.

In one embodiment, the optical means further comprise a long-pass filter placed in an fiber port of the detector to further reduce the amount of stray laser light entering the detector.

In one embodiment, the optical means comprises a delivery means for delivering the light emitted from the light source to the tissues, and a collection means for collecting the autofluorescence signals emitted from the tissues in response to the illumination.

In one embodiment, the detector comprises at least one camera, and/or a spectrometer.

In yet another aspect, the invention relates to a method for providing anatomical guidance in surgery. The method comprises illuminating tissues of a target region with light having a wavelength; obtaining autofluorescence signals emitted from the illuminated tissues in response to the illumination; and processing the obtained autofluorescence signal to identify target tissues. In one embodiment, the wavelength is in an NIR range of about 600-1500 nm.

In one embodiment, the method further comprises displaying images corresponding to the processed autofluorescence signals for surgical guidance.

In one embodiment, said processing step comprises finding maximum autofluorescence intensities and/or emission wavelength peaks from the obtained autofluorescence signals.

In one embodiment, the autofluorescence has a highest intensity in cortex of an adrenal gland as compared to that in medulla of the adrenal gland or the periadrenal fat around the adrenal gland.

In one embodiment, the intensity of the autofluorescence of endocrine tumor in the adrenal cortex is much greater than that of endocrine tumor in the adrenal medulla.

In one embodiment, the autofluorescence is strong in pancreatic islet cells relative to other tissues.

In one embodiment, said processing step comprises obtaining autofluorescence patterns from the obtained autofluorescence signals; and correlating between the autofluorescence patterns and characteristics of the tissues of the target region.

In one embodiment, said processing step further comprises, when high correlation between the autofluorescence patterns and the characteristics of the tissues of the target region exists, indicating a surgeon to selectively excise specific tissues of the target regions.

In one embodiment, said processing step further comprises, optimizing imaging parameters that enable the surgeon to specifically identify locations of the diseased tissues and the healthy tissues in the target region.

In a further aspect, the invention relates to a system for cell-sorting to improve islet cell extraction for islet cell transplantation. The system comprises an optofluidic platform comprising an inlet for tissue lysate, an outlet for inlet cell concentration, and one or more residue pools for accommodating non-inlet cells; a plurality of switches; and a plurality of microfluidic channels, each microfluidic channel having a first channel portion fluidically coupled between the inlet and a switch of the plurality of switches, a second channel portion fluidically coupled between said switch and the outlet, and a third channel portion fluidically coupled between said switch and one of the one or more residue pools, wherein said switch is configured to operably divert a flow from the first channel portion to the second channel portion, or from the first channel portion to the third channel portion.

The system further comprises a light source configured to emit light having an NIR wavelength to illuminate each single cell as it flows through the first channel portion of each microfluidic channel; and a detector configured to collect autofluorescence signal emitted from each single cell flowing through the first channel portion of each microfluidic channel, wherein when the collected autofluorescence signal by the detector are greater than a signal threshold, an electrical impulse is sent from the detector to an inverse transducer that flips said switch to divert the flow of said single cell from the first channel portion to the second channel portion, thereby causing said single cell to flow into the outlet, and when no autofluorescence is detected, said switch reverts to its original position, thereby causing non-islet cells to flow into one of the residue pools in the optofluidics platform.

In one embodiment, the light source comprises a plurality of NIR light emitting diodes (LEDs) located on the optofluidic platform, wherein each NIR LED is configured to emit the light to illuminate each single cell as it flows through the first channel portion of each microfluidic channel.

In one embodiment, the detector comprises a plurality of photodiode detectors, each photodiode detector is configured to collect autofluorescence signal emitted from each single cell flowing through the first channel portion of each microfluidic channel.

In one embodiment, the detector further comprises a plurality of long-pass filters, each long-pass filter is configured to remove out stray LED light of a respective NIR LED and to allow true autofluorescence signal emitted from said islet cell to be collected by a respective photodiode detector.

In one embodiment, the system is usable for differentiating pancreatic islet cells from other pancreatic cells without sacrificing throughput, and separating the islet cells from a tissue preparation and maximizing its yield for islet cell transplantation.

In another aspect, the invention relates to a method for cell-sorting to improve islet cell extraction for islet cell transplantation. The method comprises illumining each single cell of donor tissue with light having a NIR wavelength; collecting autofluorescence signal emitted from each single cell; and diverting said single cell to an outlet for islet cell concentration when the collected autofluorescence signal emitted from said single cell is greater than a signal threshold, or diverting said single cell to a non-islet cell pool when no autofluorescence emitted from said single cell is detected.

In one embodiment, the donor tissue is pancreatic tissue.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 shows schematically a near-infrared (NIR) imaging system according to one embodiment of the invention.

FIGS. 2A-2B show schematically a NIR spectroscopy system according to one embodiment of the invention. FIG. 2A: the NIR spectroscopy system. FIG. 2B: a cross-sectional view of the probe along lines A-A′ shown in FIG. 2A.

FIG. 3 shows schematically an optical fiber probe-based NIR fluorescence detection system according to one embodiment of the invention.

FIG. 4 shows schematically a multi-channel microfluidics-based flow-cytometry system according to one embodiment of the invention.

FIG. 5 shows NIR autofluorescence images of different endocrine tissues.

FIG. 6 shows NIR autofluorescence images of normal and malignant pancreas under 680 nm and 785 nm illumination.

FIG. 7 shows autofluorescence intensity of normal pancreas and malignant pancreas.

FIG. 8 shows white light image, NIR autofluorescence and composite images of different endocrine organs. NIR autofluorescence intensity was the strongest for adrenal and parathyroid tissue.

FIG. 9 shows NIR autofluorescence images of human pancreatic islet cells. The images were acquired with NIR (785 nm) for 30 human islets in solution, showing very high fluorescence (20,000 counts). Well was mixed to ensure signal was not an artifact. Islet cell extraction using NIR autofluorescence can be used for islet cell transplantation in conditions with insulin insufficiency—Type 1 diabetes, pancreas cancer, chronic pancreatitis.

FIG. 10 shows NIR microscopy of dried pancreatic islet cells, with 45 ms exposure time, 3 accumulations. Dried pancreatic islet cell dried in media.

FIGS. 11-49 shows NIR autofluorescence images of excised adrenal tumors acquired from patients who underwent adrenalectomy or healthy adrenal glands in patients who underwent nephrectomy. Data were obtained from over 33 patients. NIR autofluorescence intensity was the highest in adrenal glands compared to adjacent soft tissues. Intensity was the highest for cortical adenomas compared to other adrenal lesions

FIGS. 50-58 shows NIR autofluorescence images acquired from 6 patients with tumors in small bowels. Intensity was higher for neuroendocrine tumors of small bowel compared to adjacent healthy bowel.

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “autofluorescence” refers to the fluorescence produced by a molecule of interest without the use of exogenous markers. Autofluorescence may serve as a useful diagnostic indicator such as in the case of “biological autofluorescence”, which refers to the fact that cells contain molecules, which become fluorescent when excited by radiation of suitable wavelength. This fluorescence emission, arising from endogenous fluorophores, is an intrinsic property of cells and is called autofluorescence to be distinguished from fluorescence signals obtained by adding exogenous markers. The majority of cell autofluorescence originates from mitochondria and lysosomes. Together with aromatic amino acids and lipo-pigments, the most important endogenous fluorophores are pyridinic (NADPH) and flavin coenzymes. In tissues, the extracellular matrix often contributes to the autofluorescence emission more than the cellular component, because collagen and elastin have, among the endogenous fluorophores, a relatively high quantum yield. Changes occurring in the cell and tissue state during physiological and/or pathological processes result in modifications of the amount and distribution of endogenous fluorophores and chemical-physical properties of their microenvironment. Therefore, analytical techniques based on autofluorescence monitoring may be utilized in order to obtain information about morphological and physiological state of cells and tissues. Moreover, autofluorescence analysis can be performed in real time because it does not require any treatment of fixing or staining of the specimens.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

In certain aspects, the invention relates to a systems and methods for providing anatomical guidance in endocrine surgery, in a target region in a living subject. The target region can be an adrenal gland, a pancreas gland, a thyroid or parathyroid gland, a small bowel, liver or any other organ of an endocrine system of the living subject.

Specifically, one embodiment of this invention is to utilize NIR imaging to intraoperatively guide surgeon in localizing and identifying specific tissue regions in the donor pancreas quickly for islet extraction, rather than utilizing the whole organ, so as to decrease the time required for digestion of pancreatic tissue. Another embodiment of this invention is to design an optofluidic device that relies on flow cytometry with NIR autofluorescence detection to separate islets from other non-islet pancreatic tissues, so as to increase the extraction efficiency and extend the viability of the extracted islet cells. Yet another embodiment of this invention is to improve the extraction rate of islet cells from pancreatic tissue by differentiate pancreatic islet cells from other pancreatic cells/tissues without sacrificing throughput and guiding the surgeon to target/localize regions in pancreas that have maximum density of islet cells. According to the invention, the yield of the islet cells extracted from the pancreas can be suitably enhanced and be specifically harvested for transplantation using NIR autofluorescence detection.

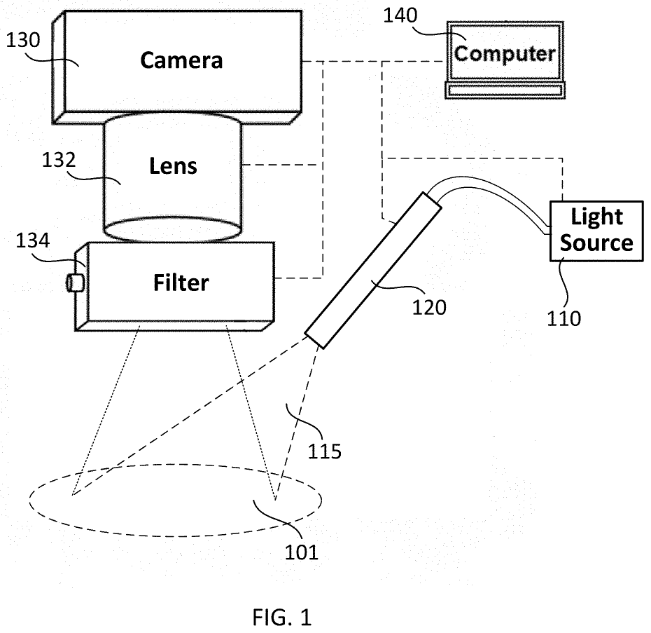

Referring to FIG. 1, the system is shown according to one embodiment of the invention. The system is adapted for providing anatomical guidance in a surgery. In one embodiment, the surgery is an endocrine surgery. In this exemplary embodiment, the system includes a light source 110 configured to emit a beam of light 115 for illuminating tissues 101 in a target region, such as, but is not limited to, pancreas; means for obtaining autofluorescence signals emitted from the illuminated tissues 101 in response to the illumination; and a controller (e.g., computer) 140 configured to process the obtained autofluorescence signals to identify target tissues. In some embodiments, identifying the target tissues comprises differentiating diseased tissues from healthy tissues within one organ, or differentiating one organ from other tissues or organs. The target region can be an adrenal gland, a pancreas gland, a thyroid gland, a parathyroid gland, small bowel, or any other organs that can emit autofluorescence, or can be differentiated by autofluorescence emitted from its surrounding tissues/organs.

The light source 110 comprises a laser or a light emitting diode (LED) configured to emit light having a wavelength in an NIR range of about 600-1500 nm.

The means for obtaining the autofluorescence signal comprises a light delivery device 120 and a detector 130. The light delivery device 120 is optically coupled with the light source 110 and configured to deliver the beam of light 115 from the light source 110 to the tissues 101 in the target region. The detector 130 is configured to collect the autofluorescence signals emitted from the illuminated tissues 101.

In one embodiment, the light delivery device 120 includes one or more light guides, or one or more optical fibers. In another embodiment, the light delivery device 120 include a wavelength appropriate notch filter and a double concave lens for beam divergence. In yet another embodiment, the light delivery device 120 may include means for focusing from the light source to the tissues of the target region. The focusing means may have optical components including, but are not limited to, one or more lenses, one or more mirrors, one or more optical splitters, etc.

As shown in FIG. 1, the means for obtaining the autofluorescence signal may also further comprise at least one lens 132 positioned between the tissues 101 and the detector 130 in an optical path along which the autofluorescence signals emitted from the illuminated tissues 101 travels, and utilized to receive the autofluorescence signals emitted from the tissues 101 and focus them on a desired spot on the detector 130. Further, the means for obtaining the autofluorescence signals may comprise at least one filter 134 positioned between the tissues 101 and the at least one lens 132 in the optical path, and utilized to block unwanted wavelengths, ambient light and/or reflected light from the illuminated tissues 101 to ensure the autofluorescence signals to be detected.

In one embodiment shown in FIG. 1, the detector 130 comprises at least one camera. In one embodiment, the at least one camera comprises at least one charge-coupled device (CCD) camera, at least one complementary metal oxide semiconductor (CMOS) camera, at least one photosensor array, or a combination thereof.

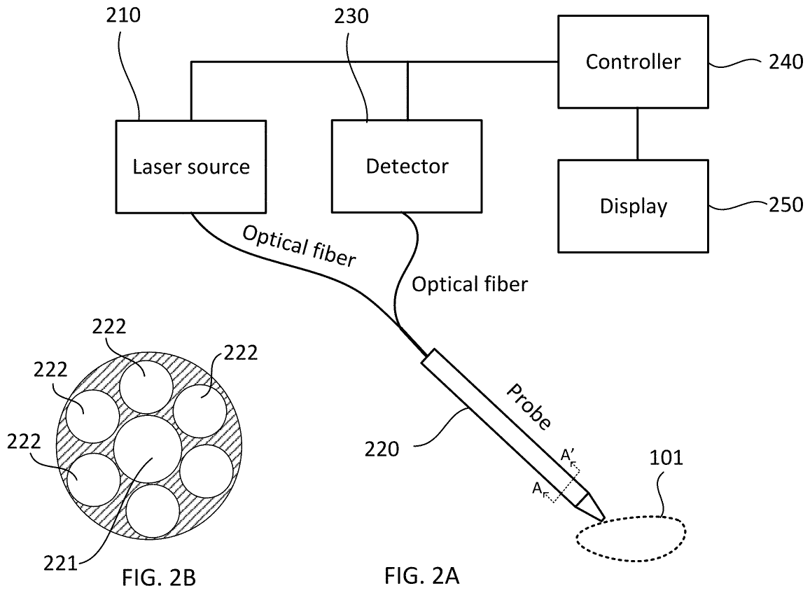

In one embodiment shown in FIG. 2, the detector 230 comprises a spectrometer.

In one embodiment, the controller 140 comprises a center processing unit (CPU) or a graphics processing unit (GPU).

In one embodiment, the controller 140 is a computer including a display for displaying images corresponding to the autofluorescence signals emitted from the illuminated tissues.

The system shown in FIG. 1 can be an NIR imaging system. The NIR imaging system is capable of capturing NIR fluorescence images from tissue in real-time that can be used intraoperatively to distinguish between healthy and cancerous/diseased tissues in endocrine abdominal organs.

In some embodiments, the NIR imaging system includes a NIR diode laser coupled via a SMA905 connector to a wavelength appropriate notch filter and a double concave lens for beam divergence. Image collection is accomplished using an NIR complementary metal-oxide-semiconductor (CMOS) camera, equipped with a c-mounted manual iris lens and a c-mounted machine vision with a wavelength-appropriate hybrid long-pass filter. The NIR imaging system as a whole is controlled with a computer such as a computer such as a laptop, running a custom MATLAB® graphical user interface (GUI) suite that utilizes the GPU for simultaneous NIR image capture and data processing. The NIR imaging system enables surgeon to target/localize regions in pancreas that have maximum density of islet cells for improving the yield for islet cell transplantation.

Data with NIR imaging system indicate strong NIR autofluorescence intensities in organs like pancreas, adrenals, liver, thyroid or parathyroid, and a small bowel.

Preliminary findings suggest that islet cells are responsible for the NIR autofluorescence in pancreas, compared to other tissues in the gastrointestinal system. In addition, preliminary study in vivo experiments indicated that a highly inflammed/fibrosed pancreas found in one patient had much lower NIR autofluorescence compared to non-inflammed pancreas in other patients. It is postulated that inflammation and subsequent fibrosis of pancreas in this one patient led to loss of islet cells and resultant decrease in the NIR autofluorescence of this pancreas as compared to pancreas from other patients.

The experiments include obtaining in vivo images of pancreas with the NIR imaging system to visualize the NIR autofluorescence patterns in pancreas. Corresponding histology/microscopy of these specimens are evaluated to correlate between the NIR autofluorescence patterns in the image and regions of high islet concentration in the specimens. High correlation between the NIR autofluorescence pattern and islet cell distribution indicates that surgeons can use this technique to selectively excise specific regions of the pancreas for maximizing islet extraction process.

Preliminary imaging data obtained from ex vivo pancreas specimens indicate that malignant pancreas specimens have lower NIR autofluorescence than healthy pancreas, which has potential for tumor margin guidance.

Referring to FIGS. 2A-2B, the system is shown according to another embodiment of the invention. Similarly, the system can be used for providing anatomical guidance in a surgery, such as, but is not limited to, an endocrine surgery. The system includes a light source 210 configured to emit a beam of light; an optical means 220 optically connected to the light source 210 and configured to deliver the emitted beam of light to tissues 101 in the target region to illuminate the tissues 101, and to collect light of autofluorescence signals emitted from the tissues in the pancreas in response to the illumination; a detector 230 optically coupled with the optical means and configured to obtain the collected autofluorescence signals; and a controller 240 in communication with the detector 230 and configured to process the obtained autofluorescence signals to identify target tissues. In some embodiments, the identifying the target tissues comprises differentiating diseased tissues from healthy tissues within one organ, or differentiating one organ from other tissues or organs.

The light source 210 comprises a laser or an LED configured to emit light having a wavelength in a NIR range of about 600-1500 nm.

The optical means 220 comprises an excitation fiber 221 configured to deliver the beam of light emitted from the light source 210 to the tissues 101 in the target region, and a plurality of collection fibers 222 aligned circularly around the excitation fiber 221 (FIG. 2B) configured to collect the autofluorescence signal emitted from the tissues in the pancreas in response to the illumination.

In one embodiment, the optical means 220 further comprises an inline filter for preventing intrinsic autofluorescence from the optical fiber itself. In one embodiment, the optical probe further comprise a long-pass filter placed in an fiber port of the detector to further reduce the amount of stray laser light entering the detector 230.

In another embodiment, the optical means comprises a delivery means for delivering the light emitted from the light source to the tissues, and a collection means for collecting the autofluorescence signals emitted from the tissues in response to the illumination. In one embodiment, the delivery means can include fibers, or the like. In another embodiment, the delivery means can include one or more lenses, one or more mirrors, and/or one or more splitters. In one embodiment, the collection means can include fibers, or the like. In another embodiment, the collection means can include one or more lenses, one or more mirrors, and/or one or more splitters.

In one embodiment, the detector 230 comprises a spectrometer. In another embodiment, the detector 230 comprises one or more CCD cameras, one or more CMOS cameras, or one or more photosensor array.

The system shown in FIG. 2A-2B can be an NIR autofluorescence spectroscopy system, or an optical fiber probe-based NIR fluorescence detection system.

The NIR spectroscopy system is capable of detecting NIR fluorescence spectra from tissue in real-time, which can be used intraoperatively to distinguish between healthy and cancerous/diseased tissues in endocrine abdominal organs.

In some embodiments, the NIR spectroscopy system includes a NIR diode laser that is delivered to the tissue surface with a fiber-optic probe that has a spot size of 400 μm. Tissue fluorescence spectra that is detected with the fiber-optic probe is relayed to a spectrometer. Spectral acquisition is computer controlled by a custom-designed software developed in LabView® (National Instruments, Austin, Tex.). The fiber-optic probe includes 7 collection fibers aligned circularly around 1 excitation fiber. Inline filtering in the probe prevents intrinsic NIR autofluorescence from the optical fiber itself. Additional 3-mm diameter long-pass filter was placed in the fiber port of the spectrometer to further reduce the amount of stray laser light entering the detector. Acquired data is eventually output as fluorescence spectra of the tissue.

The optical fiber probe-based NIR fluorescence detection system is designed originally to detect NIR autofluorescence for identifying parathyroid glands in the neck. However, the scope of this system to rely on NIR autofluorescence detection to differentiate between healthy and diseased/cancerous tissues during abdominal endocrine surgeries has not been explored or investigated yet. The novelty of the technique being disclosed here involves using/applying a commercial optical fiber probe-based device such as PTeye (AiBiomed, now acquired by Medtronic) for the first time to guide endocrine surgeries in abdomen to (i) differentiate one endocrine organ from its surrounding tissues and adjacent organs (ii) differentiate between healthy and cancerous/diseased tissues in endocrine abdominal organs and (iii) identify regions of localized cancer spread from a diseased endocrine organ in the abdomen. The measured fluorescence counts detected with the device provides a quantitative output to distinguish/identify diseased tissue from healthy regions.

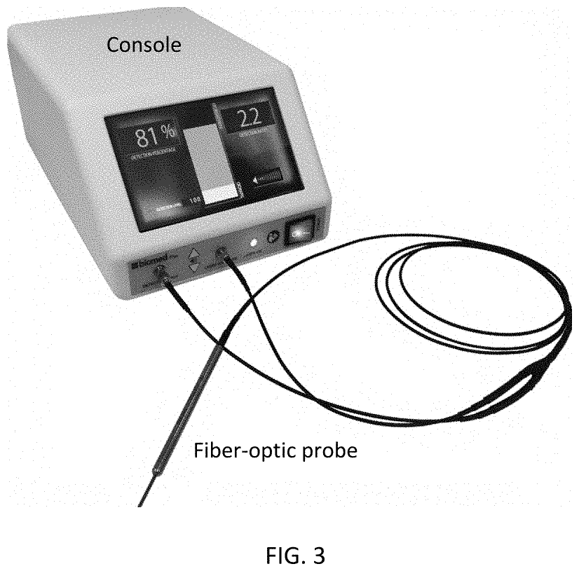

PTeye (originally licensed between Vanderbilt University and AiBiomed, now purchased by Medtronic) that is a commercially developed version of the lab-built NIR spectroscopy system (FIGS. 2A-2B) originally designed for parathyroid identification in the neck, as shown in FIG. 3 includes four components: a console, a fiber-optical probe, a foot pedal, and a power cord. The fiber-optic probe is connected to the front of the console via two optical connectors. The foot pedal is connected to the back of the console via Lemo connector. The power supply is connected to the back of the console. The power button on the front of the console powers the system on.

The Console:

The console includes a LED display that provides information on the operation of the system including indication that the laser is on. The main components of the console are an LED indicator that indicates if the laser is on, a display for visual feedback for the data collected, a speaker for auditory feedback, a photo detector, a 785 nm laser diode module—20 mW output, and two circuit boards with microprocessor. The console is 5.5 inches in height by 8.5 inches in width by 13 inches in length, and includes the capability for an SD card and USB connection.

The Fiber-Optic Probe:

The fiber-optic probe assembly is composed of optical elements that allow for transmission of light and collection of emitted fluorescence. The components of the fiber optical probe assembly can be broken down into 3 sections: the probe tip, the probe body and the connectors. The overall length of the fiber optic probe is about 2500 mm (98.4 inches).

The probe tip includes the following components. (1) A transmission optical fused silica fiber that emits the 785 nm wavelength to the tissue. This full-length 600 μm fused silica glass fiber has a long pass filter secured on one end with Loctite 3311 adhesive. (2) A collection optical fused silica fiber that collects the desired wavelength (peak at 822 nm) from the tissue. This full-length 300 μm fused silica glass fiber has a band pass filter secured on one end with Loctite 3311 adhesive. (3) 316 stainless steel hypo tubing used to align the band filter in the fiber tip. (4) EPO-TEK 301, a medical grade epoxy that is used to secure all the optical components in the probe tip and create a seal. (5) A disposable plastisol protective cap on the probe tip that is removed prior to use. (6) 316 stainless steel needle tube used as the probe tip to house all the optical components. (7) 304 stainless steel tube placed over the fiber tip to function as the probe handle.

The probe body includes the continuation of combined fused silica fibers extends from the probe tip to the acrylonitrile butadiene styrene (ABS) “Y” connector. After the “Y” connector, the individual fibers are split and secured to the optical connectors. Each single fiber is encased in Teflon protective tubing and covered jointly and independently in acrylate olefin heat shrink tubing.

The connectors include the following components. The 300 μm fiber is terminated and secured to a SMA905 connector using EPO-TEK 301 epoxy. The 600 μm fiber is terminated and secured to a FC/PC connector using EPO-TEK 301 epoxy.

The Foot Pedal:

The CE marked and UL approved foot pedal connects to the back of the console via a Lemo connector.

The External Power Supply and Power Cord:

The power cord connects to the back of the console and is 8.2 feet (2.5 meters) long and rated as 125V 10 Amp. Uses a listed (UL, CSA) detachable power cord. A ferrite bead has been added to the external power supply cord. It is located 50 millimeters from the input end (console) of the cord.

The above described technologies can be used for (i) tumor differentiation from healthy tissues in endocrine organ surgeries, (ii) tissue margin assessments of abdominal endocrine organs and (iii) identify regions of localized cancer spread from a diseased endocrine organ in the abdomen. Therefore, they can be used for providing anatomical guidance in endocrine surgery.

One aspect of the invention also disclose such a method of using NIR autofluorescence for providing anatomical guidance in a surgery. Specifically, the method comprises illuminating tissues of a target region in a living subject with light having a predetermined wavelength in as NIR range of about 600-1500 nm; obtaining autofluorescence signals emitted from the illuminated tissues in response to the illumination; processing the obtained autofluorescence signal to identify target tissues; and displaying images corresponding to the processed autofluorescence signal for surgical guidance.

In some embodiments, said processing step comprises finding maximum autofluorescence intensities and/or emission wavelength peaks from the obtained autofluorescence signal.

In some embodiments, the NIR autofluorescence has a highest intensity in cortex of an adrenal gland as compared to that in medulla of the adrenal gland or the periadrenal fat around the adrenal gland.

In some embodiments, the intensity of the NIR autofluorescence of endocrine tumor in the adrenal cortex is much greater than that of endocrine tumor in the adrenal medulla.

In some embodiments, the NIR autofluorescence is strong in pancreatic islet cells relative to other tissues.

In some embodiments, said processing step comprises obtaining NIR autofluorescence patterns from the obtained autofluorescence signal; and correlating between the NIR autofluorescence patterns and characteristics of the tissues of the target region.

In some embodiments, said processing step further comprises, when high correlation between the NIR autofluorescence patterns and the characteristics of the tissues of the target region exists, indicating a surgeon to selectively excise specific tissues of the target regions.

In some embodiments, said processing step further comprises, optimizing imaging parameters that enable the surgeon to specifically identify locations of the diseased tissues and the healthy tissues in the target region.

NIR imaging systems (e.g., Fluobeam-800 and LX, PDE-Neo II, Firefly, Karl Storz Opal-1, Quest Spectrum, PinPoint, SPY, ENV, SPY-PHI, 1588 AIM, FLARE, IMAGE1 SPIES, EleVision, Olympus) are available commercially. But all rely on contrast agents/dyes for imaging. None of these technologies have demonstrated/presented any results using NIR autofluorescence detection (i.e., without any labels/contrast agents/dyes) for guiding abdominal endocrine surgeries, particularly, for guiding islet cell transplantation/extraction from pancreas in a label-free/contrast-free manner. Fluobeam-800 and LX have been utilized NIR autofluorescence detection for parathyroid identification like PTeye, but have not applied the same for guiding abdominal endocrine surgeries.

To the best knowledge of the inventors, NIR autofluorescence detection has not been reported for guiding abdominal endocrine surgeries in a label-free manner, as most studies have always relied on systems that require exogenous contrast agents/dyes administration. The systems disclosed in this disclosure are the first that relies on NIR autofluorescence detection (i) to sort cells on an optofluidic platform for separating pancreatic islet cells from non-islet cells and (ii) guiding the surgeon to target/localize regions in pancreas that have maximum islet cell density for improving extraction yield of islet cells from pancreatic tissue. The three systems, NIR spectroscopy system, NIR imaging system and/or PTeye, can be used in abdominal surgeries for tumor excision, which includes pancreatectomies, adrenalectomies, gastrectomies, liver resection among the various others.

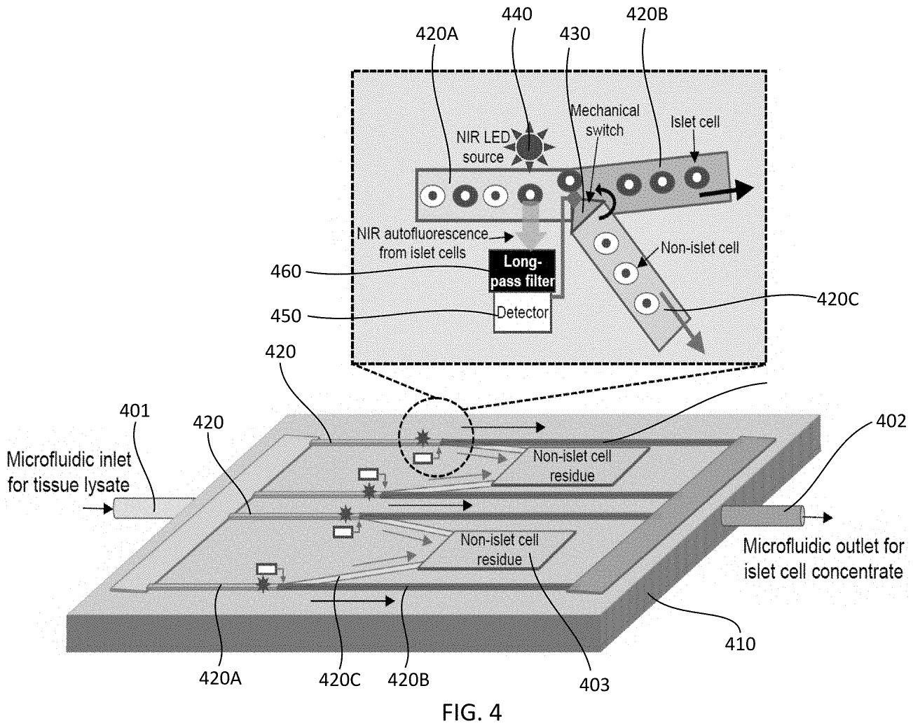

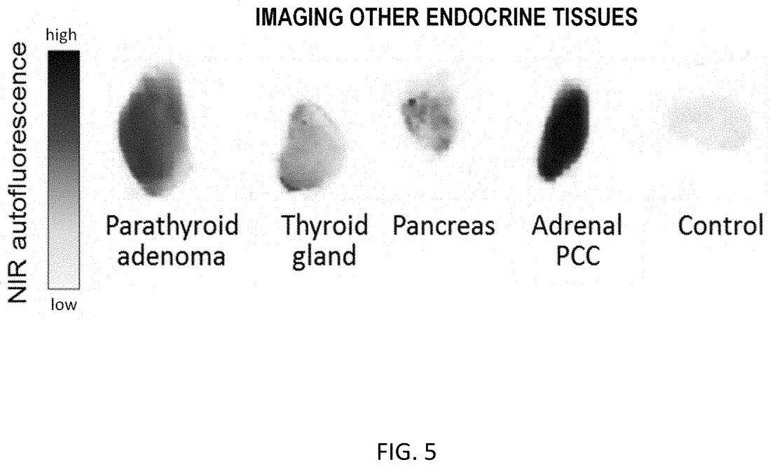

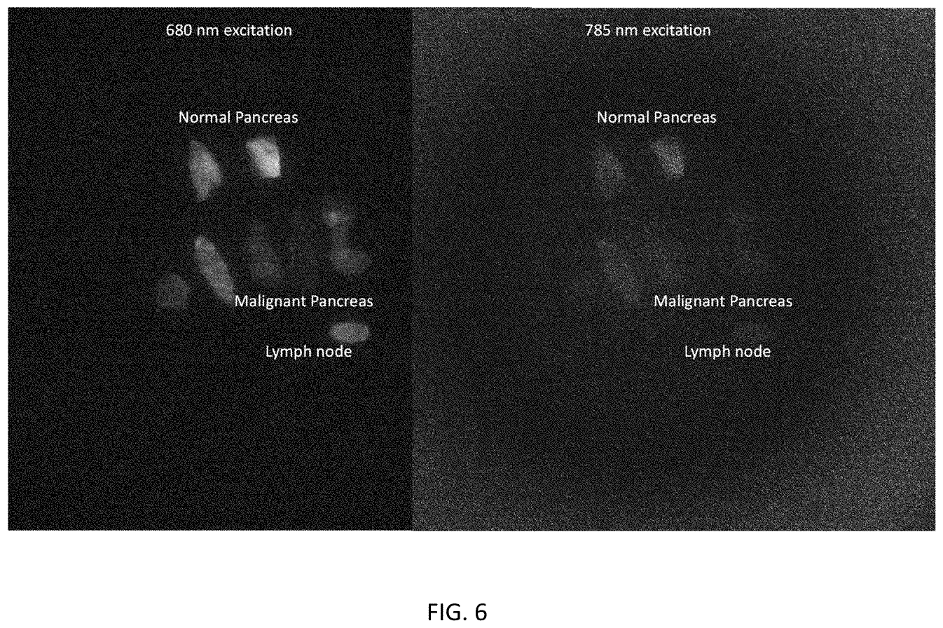

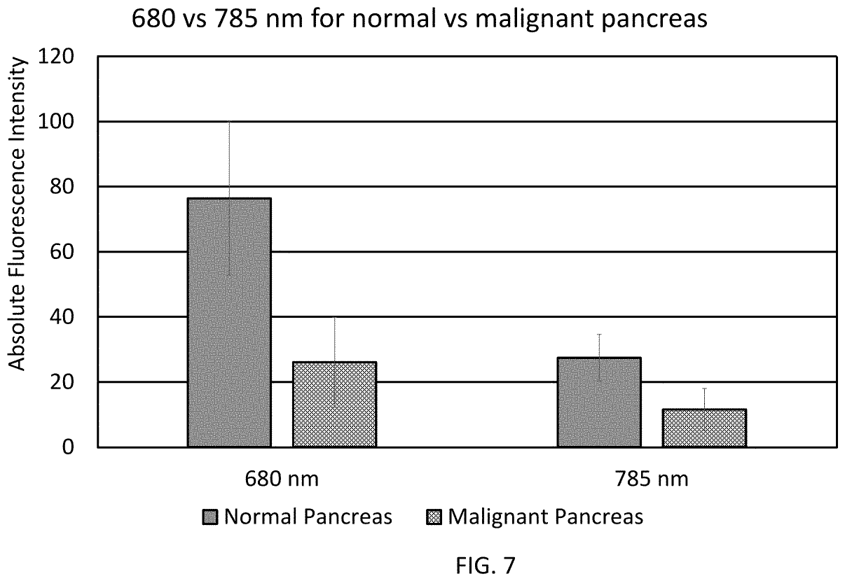

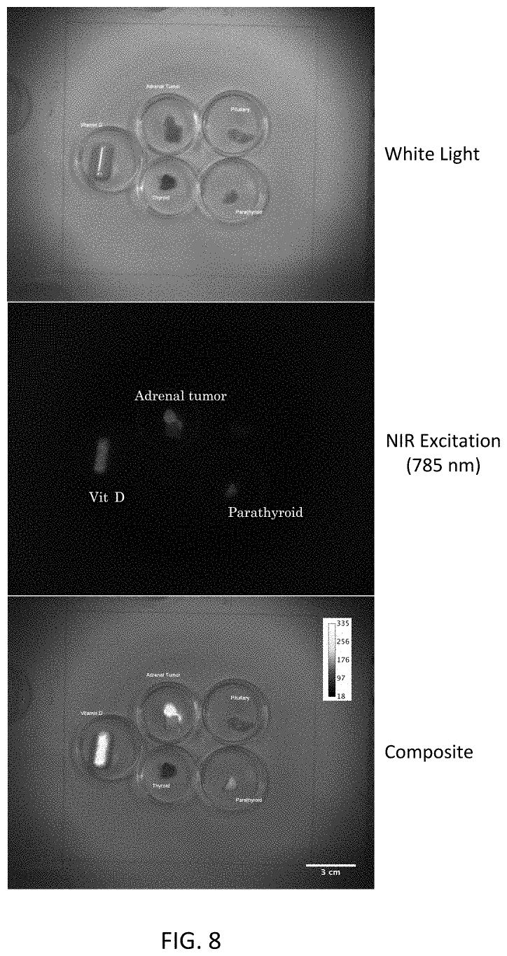

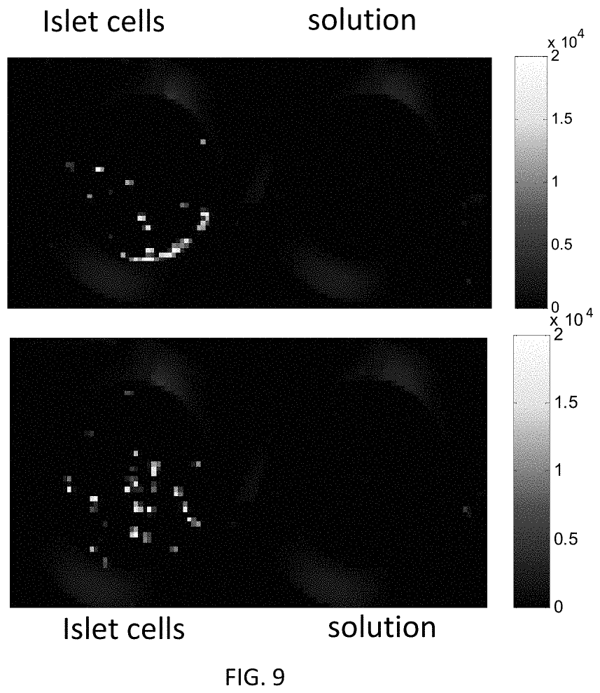

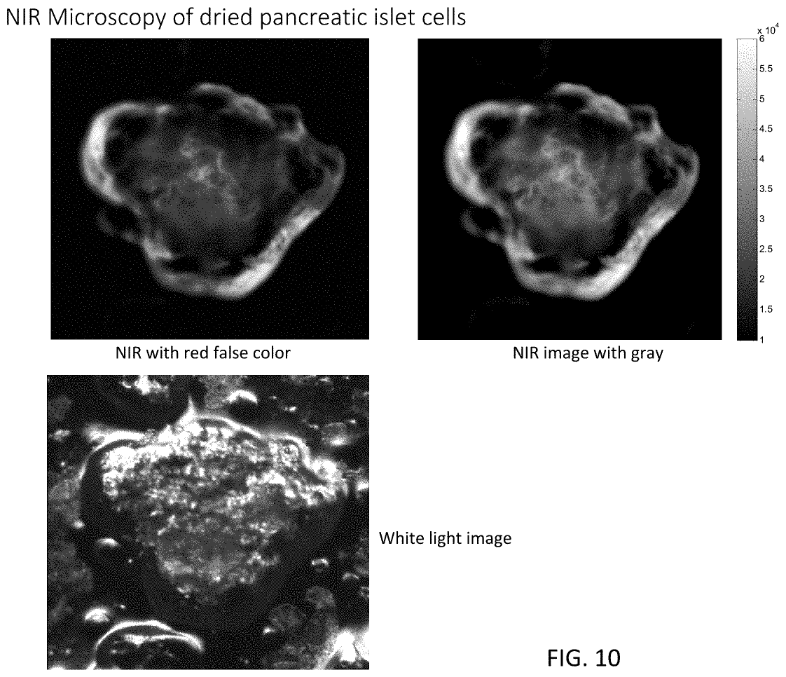

In a further aspect, the invention relates to a system for cell-sorting to improve islet cell extraction for islet cell transplantation. Referring to FIG. 4, the system is shown according to one embodiment of the invention. The system includes an optofluidic platform 410 comprising an inlet 401 for tissue lysate, an outlet 403 for inlet cell concentration, and one or more residue pools 403 for accommodating non-inlet cells; a plurality of switches 430; and a plurality of microfluidic channels 420, each microfluidic channel 420 having a first channel portion 420A fluidically coupled between the inlet 401 and a switch 430 of the plurality of switches, a second channel portion 420B fluidically coupled between said switch 430 and the outlet 402, and a third channel portion 420C fluidically coupled between said switch 430 and one of the one or more residue pools 403. Said switch 430 is configured to operably divert a flow of cells from the first channel portion 402A to the second channel portion 420B, or from the first channel portion 420A to the third channel portion 420C.

The system further comprises a light source 440 configured to emit light having an NIR wavelength to illuminate each single cell as it flows through the first channel portion 420A of each microfluidic channel 420; and a detector 450 configured to collect autofluorescence signal emitted from each single cell flowing through the first channel portion 420A of each microfluidic channel. When the collected autofluorescence signal by the detector 450 are greater than a signal threshold, an electrical impulse is sent from the detector 450 to an inverse transducer that flips said switch 430 to divert the flow of said single cell from the first channel portion 420A to the second channel portion 420B, thereby causing said single cell to flow into the outlet 402, and when no NIR autofluorescence is detected, said switch 430 reverts to its original position, thereby causing non-islet cells to flow into one of the residue pools 403 in the optofluidics platform 410.

In one embodiment, the light source 440 comprises a plurality of NIR LEDs located on the optofluidic platform 410. Each NIR LED 440 is configured to emit the light to illuminate each single cell as it flows through the first channel portion 420A of each microfluidic channel 420.

In one embodiment, the detector 450 comprises a plurality of photodiode detectors, each photodiode detector is configured to collect autofluorescence signal emitted from each single cell flowing through the first channel portion 420A of each microfluidic channel 420.

In one embodiment, the detector 450 further comprises a plurality of long-pass filters 460. Each long-pass filter 460 is configured to remove out stray LED light of a respective NIR LED and to allow true NIR autofluorescence signal emitted from said islet cell to be collected by a respective photodiode detector.

In one embodiment, the system is usable for differentiating pancreatic islet cells from other pancreatic cells without sacrificing throughput, and separating the islet cells from a tissue preparation and maximizing its yield for islet cell transplantation.

In another aspect, the invention relates to a method for cell-sorting to improve islet cell extraction for islet cell transplantation. The method comprises illumining each single cell of donor tissue with light having a NIR wavelength; collecting autofluorescence signal emitted from each single cell; and diverting said single cell to an outlet for islet cell concentration when the collected autofluorescence signal emitted from said single cell is greater than a signal threshold, or diverting said single cell to a non-islet cell pool when no NIR autofluorescence emitted from said single cell is detected.

In one embodiment, the donor tissue is pancreatic tissue.

In some embodiments, a system of guiding islet cell transplantation includes a multi-channel microfluidics-based flow-cytometry device that allows to differentiate pancreatic islet cells from other pancreatic cells without sacrificing throughput. This multi-channel optofluidic device is based on the NIR autofluorescence detection for separating the islet cells from the tissue preparation and maximizing its yield for islet cell transplantation. NIR light emitting diodes (LEDs) located on the optofluidic platform is adapted to excite single cell as it flows through the microfluidic channel. The resulting NIR autofluorescence from the islet cells flowing through the channel passes through a long-pass filter that removes out stray LED light and allows only true NIR autofluorescence signal to be collected by a miniature photodiode detector. When an adequate signal threshold is met in the detector, an electrical impulse is sent from the photodiode detector to an inverse transducer that flips a mechanical switch to a specific channel meant exclusively for islet cells. When no NIR autofluorescence is detected, the mechanical switch reverts to its original position, causing non-islet cells to flow into the residue pool in the optofluidics collection chamber.

The system can be utilized for extraction of islet cells from pancreas and optical characterization of the islet cell extract and cell sorting to improve separation of islet cells.

Benefit to Society:

Islet cell transplantation is a critical treatment for people with pancreatic insulin insufficiency including pancreatic cancer, forms of pancreatitis, and Type 1 diabetes, the latter of which affects a total of 1.35 million Americans. Improving the efficiency of islet extraction would aid in the further development of this treatment which would in turn reduce costs. Increase in islet extraction efficiency would also mean fewer transplants are required per patient and improved clinical outcomes for those undergoing islet transplantation. For people living with pancreas cancer or chronic pancreatitis, this innovation could enable them to retain their insulin producing capability, despite have their pancreas removed, which is the only guaranteed forms of treatment. More importantly, diabetic patients stand to gain the most by eliminating their dependency on insulin and the fear of sudden swings in glycemic levels. The treatment of these conditions would alleviate an enormous load on the American healthcare system.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Evaluate NIR Imaging for Surgical Guidance to Optimize Pancreatic Islet Extraction

Optimize Optical Detection of NIR Autofluorescence in Pancreatic Tissues:

Optical characteristics of pancreas may vary depending on its inherent tissue structure as well as the manner in which the organ is harvested and stored. To account for these differences, human pancreas—diseased and healthy—are optically characterized using both an NIR imaging and an NIR spectroscopy system that have been designed in house. Fluorescence images and emission spectra are collected from the tissue in five different locations to quantify the signal-to-noise (SNR) ratio. The data are compared between tissues for maximum fluorescence intensity and emission wavelength peaks. Different NIR wavelengths are investigated in combination with a variety of emission (long-pass) filters to determine which configuration gives the strongest SNR ratio. Similarly, islets cells and other type of pancreatic cells (acinar cells) harvested from the organ can be optically characterized with these systems. Preliminary results suggest that NIR autofluorescence is strong in pancreatic islet cells relative to other tissues.

Test Surgical Guidance System Classification Accuracy:

The NIR imaging system, originally designed for intraoperative surgical guidance, is modified to account for the parameters determined to optimize optical detection of NIR autofluorescence in pancreatic tissues as mentioned above. During pancreatectomy procedures at Vanderbilt University Medical Center, in vivo images of pancreas are obtained with the described NIR imaging system to visualize the NIR autofluorescence patterns in the imaged tissue. Corresponding histology/microscopy of these specimens is evaluated to correlate between the NIR fluorescence patterns in the image and regions of high islet concentration in the specimens. High correlation between NIR autofluorescence pattern and islet cell distribution would indicate that surgeons can use this technique to selectively excise specific regions of the pancreas visualized with the NIR imaging system for islet extraction process. The yield of islet cells extracted per gram of excised pancreatic tissue are compared with and without the use of NIR imaging system by the surgeon. The characterization of autofluorescence disclosed above further allows optimizing imaging parameters that can enable surgeons to specifically identify islets locations in pancreas for quick harvesting prior to transplantation.

### Example 2

### Physicochemical Characterization of Pancreatic Autofluorescence to Improve Islet Extraction

Physicochemical characterization of the fluorophore involves assessing the changes in NIR autofluorescence in pancreas tissue when subject to a variety of conditions, (i) change in temperature—fresh vs boiled vs frozen specimens, (ii) pH fluctuations—incubating pancreatic tissues in alkaline to acidic buffer solutions, (iii) solubility of fluorophore—specimens are powdered and then dissolved in a range of polar and non-polar agents to determine the best solvent for extracting this fluorophore. Biological characterization involves studying the changes in NIR autofluorescence with (i) tissue degradation—by incubating with different types of proteolytic enzymes—proteinase K, collagenase and (ii) pancreatic disease—NIR autofluorescence from pancreas is compared for healthy vs inflammatory vs cancerous conditions. Understanding the physicochemical and biological properties of the endogenous fluorophore responsible for NIR autofluorescence in the pancreas can be valuable to further improve the SNR of islet cells.

### Example 3

### Design Separation Technique Using NIR Spectroscopy

Islet cell transplantation is a key treatment strategy adopted to treat insulin insufficiency due to permanent pancreatic damage arising from tissue trauma, tumor, inflammation, or Type ½ diabetes-related changes. Conventional approaches of islet cell extraction do not have an adequate yield with an extraction rate often <50%. It is found that the human pancreas exhibits autofluorescence when illuminated with near-infrared (NIR) light. The islet cells could be the source of the NIR autofluorescence in the pancreas. This unique property of the islet cells could be exploited to maximize the yield of pancreatic islet cells being harvested for transplantation.

This exemplary study addresses the aforementioned challenges by disclosing systems and methods of using near-infrared (NIR) autofluorescence to guide endocrine surgeries and islet cell transplantation.

Design Optofluidic Platform for Cell Sorting:

Microfluidic platforms have been shown to be useful for cell analysis and sorting on a cell-by-cell basis. A multi-channel microfluidics-based flow-cytometry system could allow for the differentiating cell types without sacrificing throughput. FIG. 1 depicts the schematic of a multi-channel NIR based optofluidics that is used to separate islet cells from the tissue preparation and maximize its yield. NIR light emitting diodes (LED) are adapted to excite single cell as it flows through the microfluidic channel. The resultant NIR autofluorescence from the islet cells flowing through the channel passes through a long-pass filter that removes out stray LED light and allows only true NIR autofluorescence signal to be collected by the detector. When an adequate signal threshold is met in the detector, an electrical impulse is sent from the detector to an inverse transducer that ‘flips’ a mechanical switch to a specific channel meant exclusively for islet cells (see FIG. 4). When no NIR autofluorescence is detected, the mechanical switch reverts to its original position, causing non-islet cells to flow into the residue pool in the optofluidics collection chamber. The sorting method using this technique result in fewer islet cells being damaged and therefore more viable islets being extracted.

Test Islet Cell Yield with the Designed Optofluidic Setup:

Ten healthy pancreas specimens are obtained from Vanderbilt Cooperative Health Tissue Network tissue bank. Five of these specimens undergo enzymatic digestion and differential centrifugation using the traditional approach for islet cell extraction. The remaining 5 tissues undergo enzymatic digestion and the resulting tissue lysates undergo cell-sorting in the microfluidics setup, as shown in FIG. 4. The islet cell extracts collected from the NIR-based microfluidics setup are then compared to that from the traditional approach using (i) Dithizone staining—specific stain to quantify number of islet cells in extract and (ii) Alamar Blue assays—to assess islet cell viability. This optofluidics approach generates an islet cell extraction rate exceeding 50%, as compared to traditional approaches.

### Example 4

### Near Infrared Autofluorescence (NIRAF) from Patients Accrued with Adrenal Tumors

This exemplary study was pursued in 33 patients who had adrenal tumors. Near infrared autofluorescence (NIRAF) is significantly elevated in the adrenal glands as compared to its surroundings (fat, bowels). NIRAF was found to be the highest in cortex of adrenal gland as compared to medulla of adrenal gland or the periadrenal fat around it. NIRAF of endocrine tumor arising from adrenal cortex (e.g., cortical adenomas from Cushing's syndrome, Conn's syndrome) were significantly higher/brighter than that of endocrine tumors arising from the adrenal medulla (e.g., pheochromocytoma). NIRAF is significantly elevated in healthy adrenals as well as endocrine tumor of adrenal glands. NIRAF is notably lower in adrenal cysts and non-endocrine tumors of adrenal.

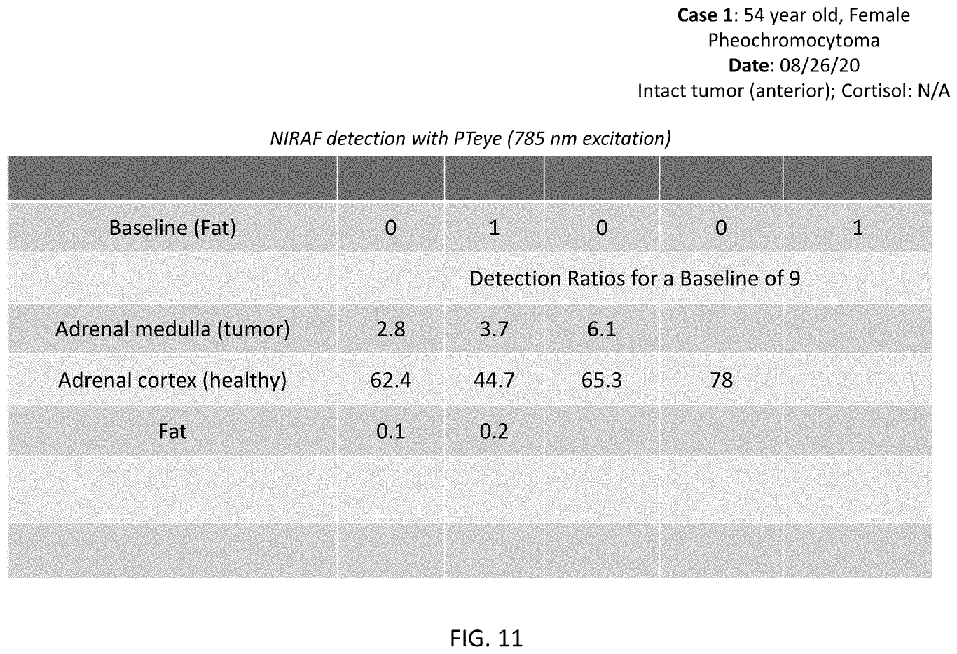

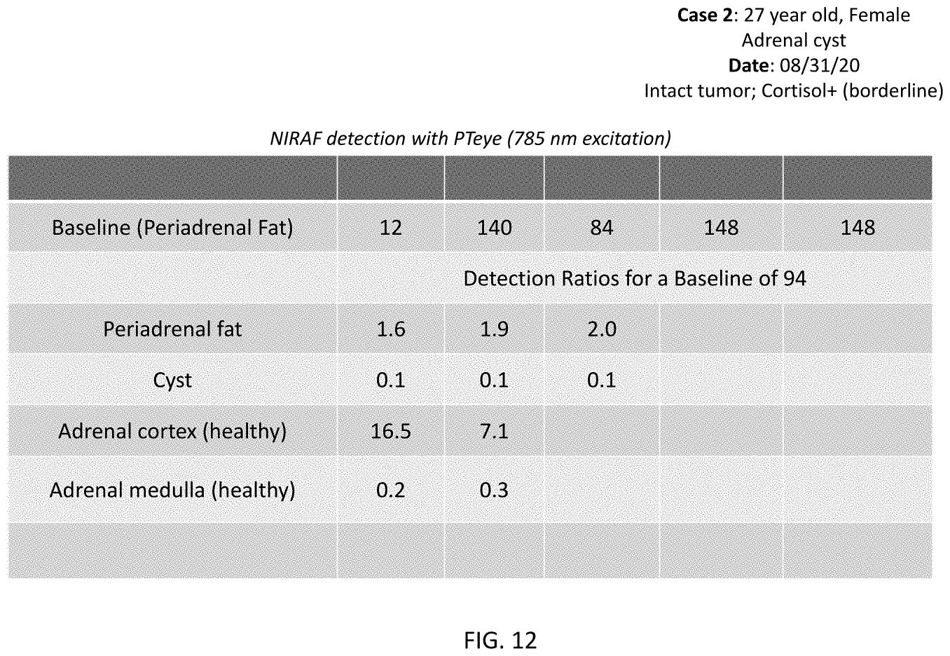

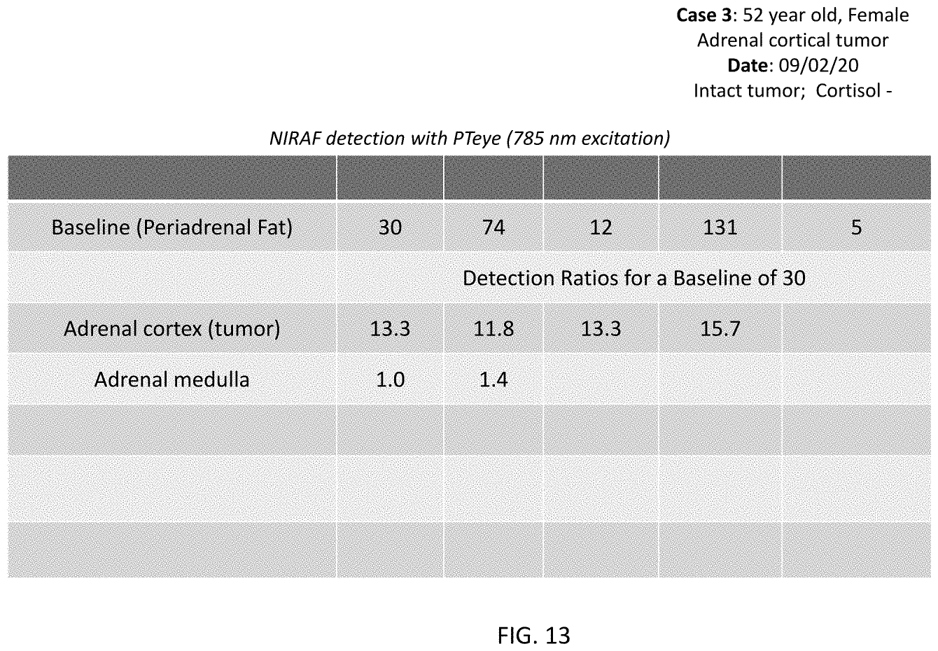

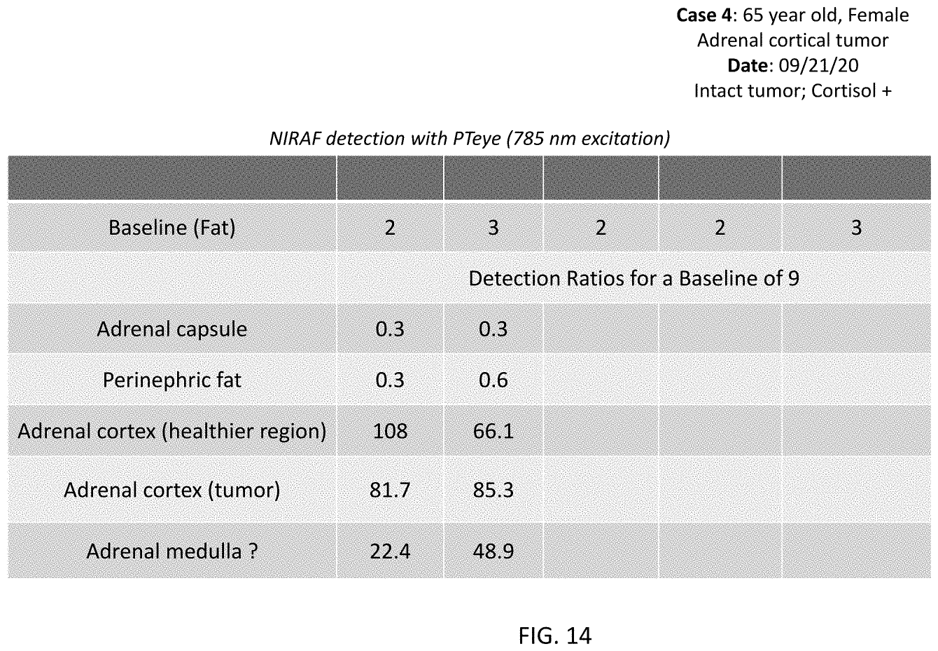

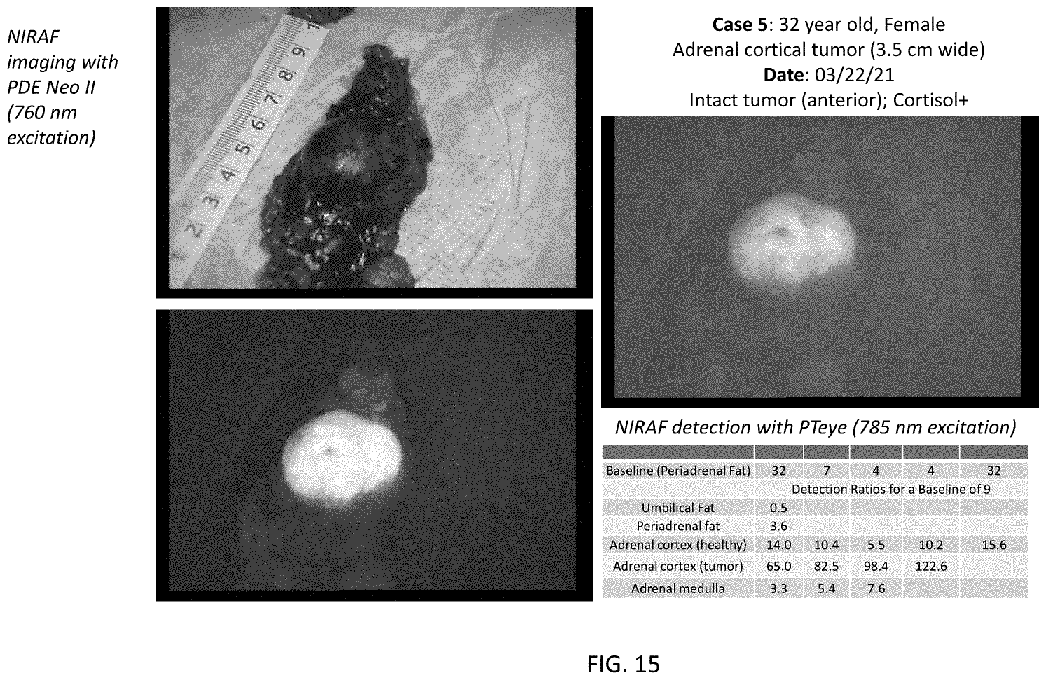

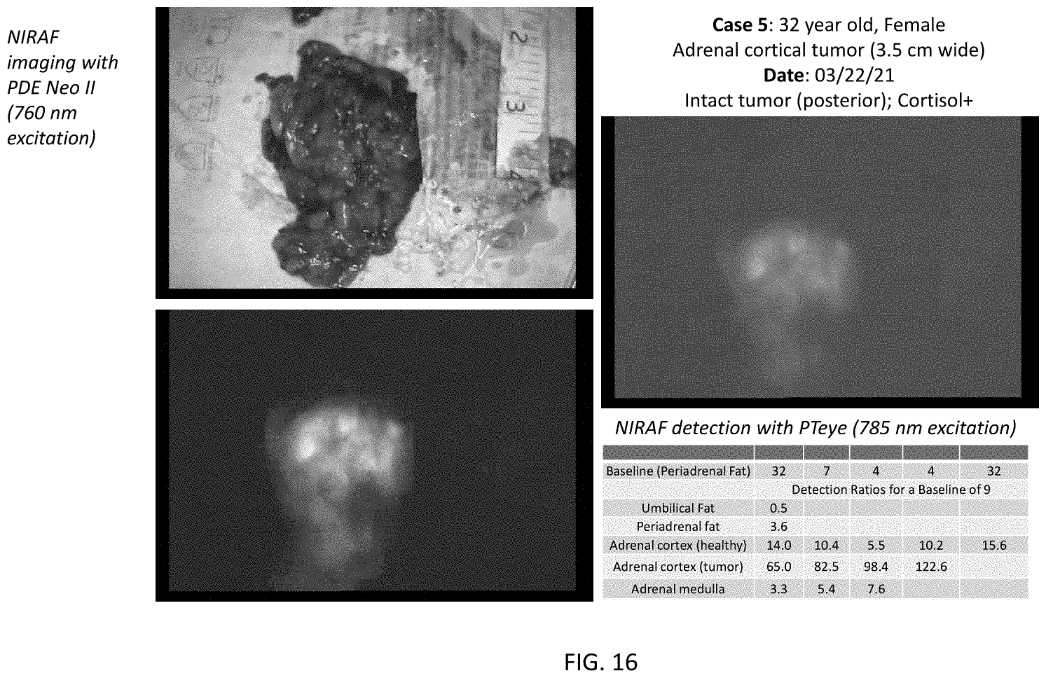

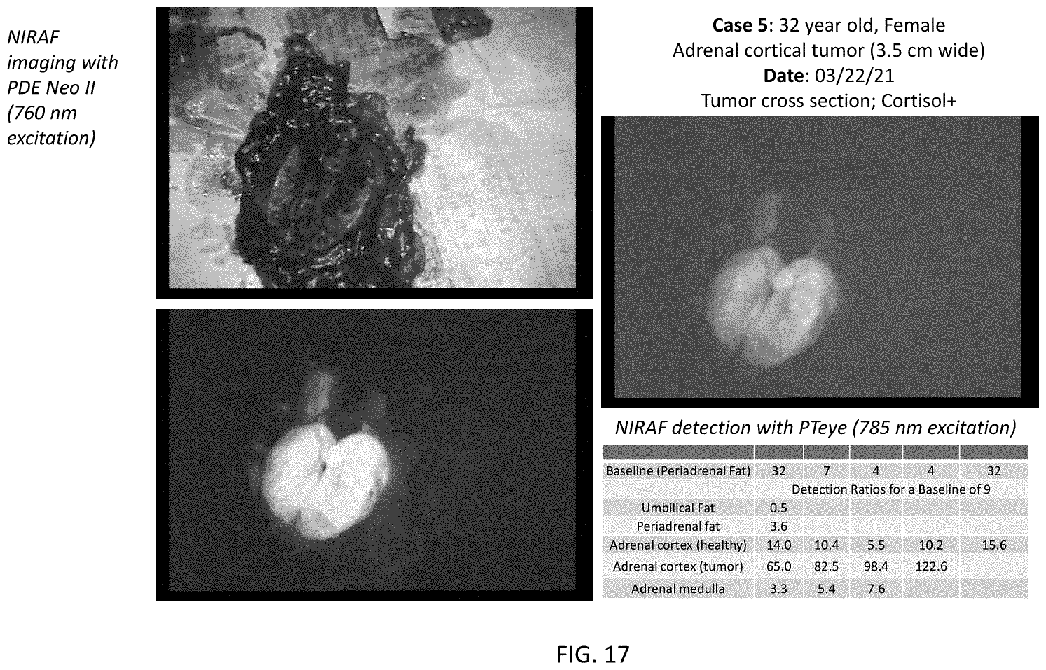

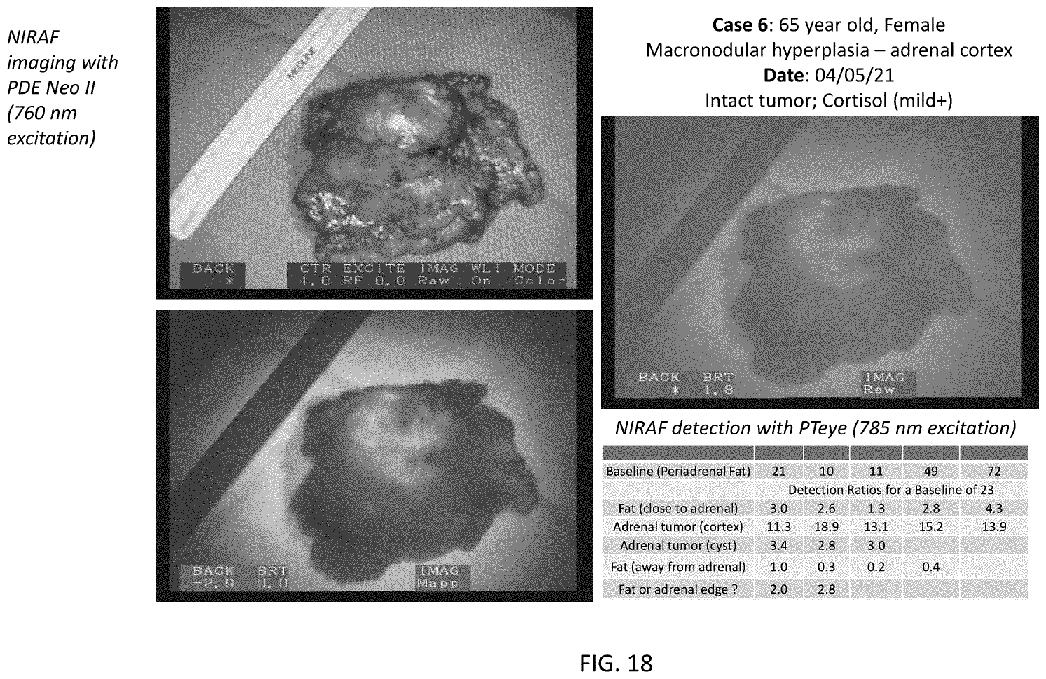

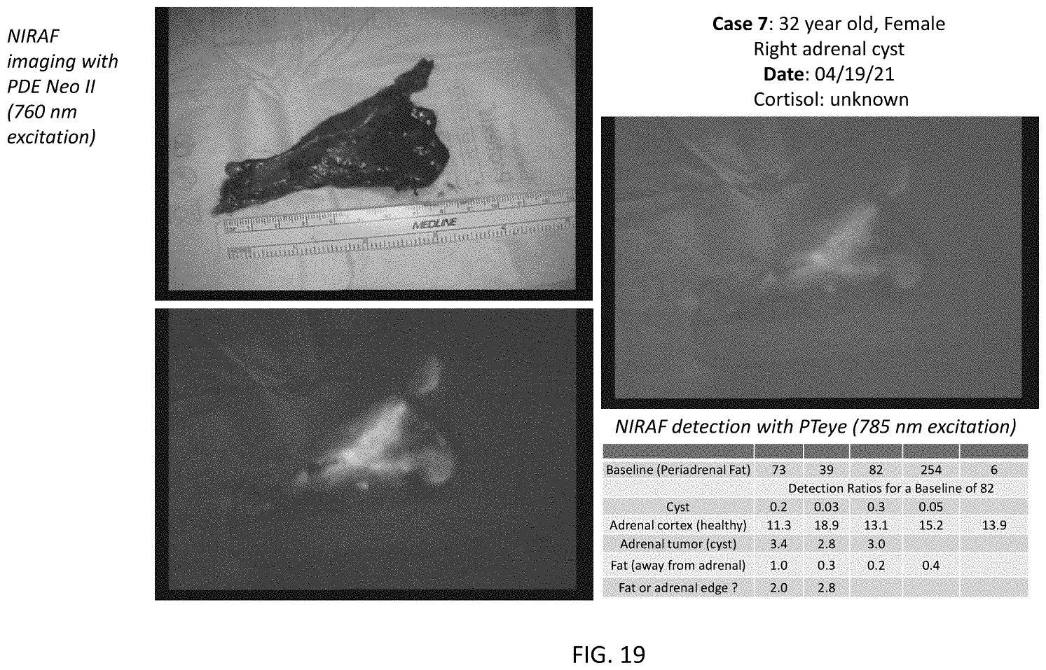

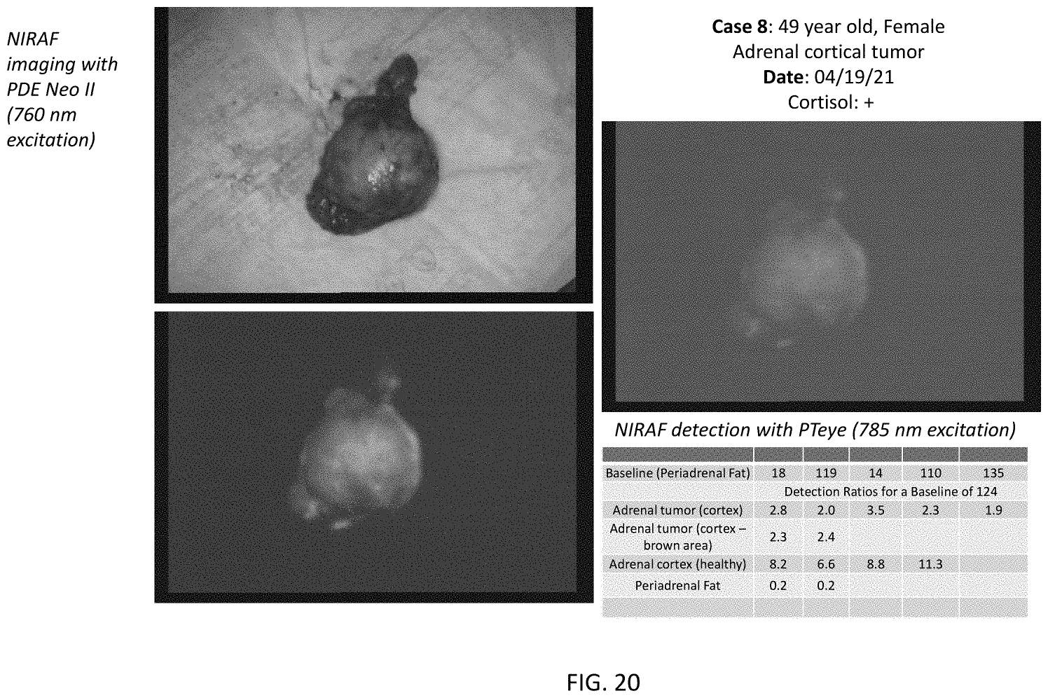

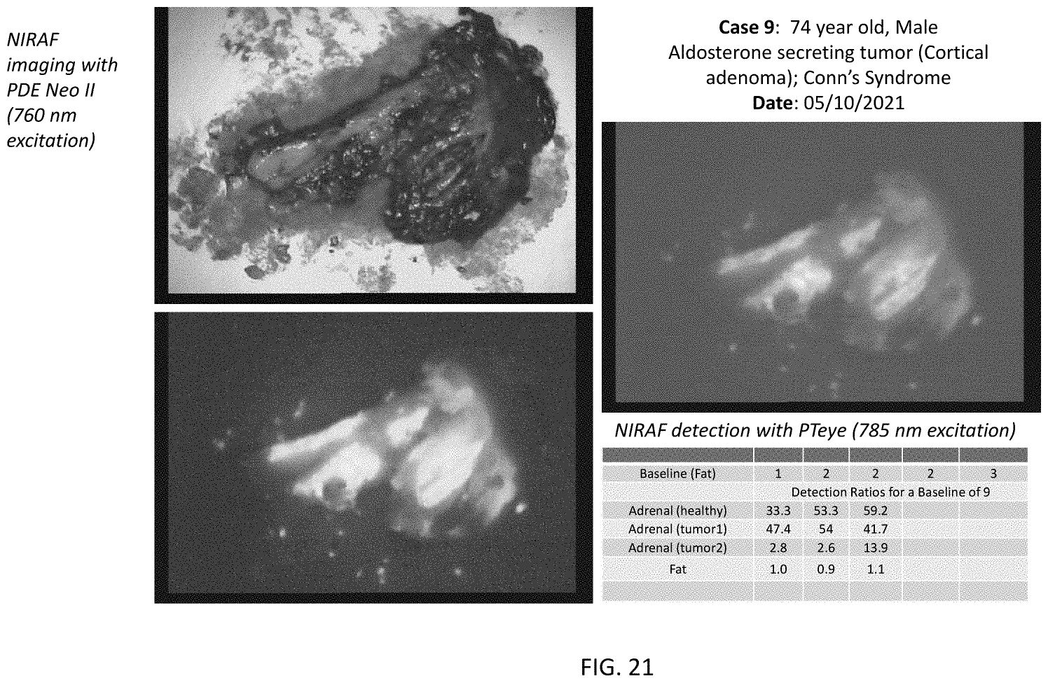

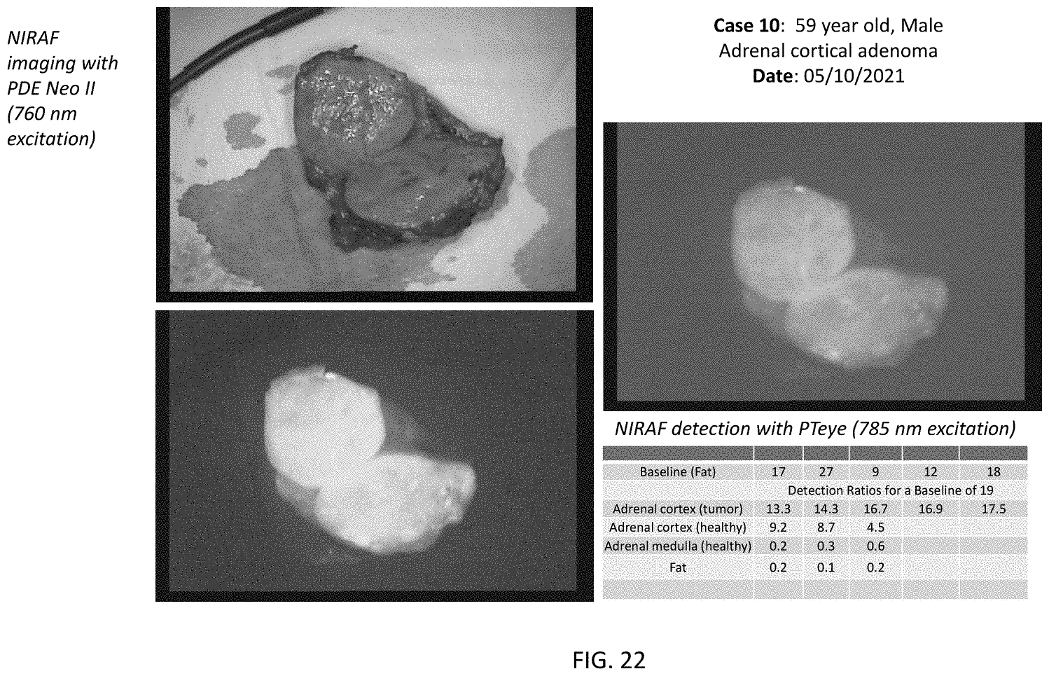

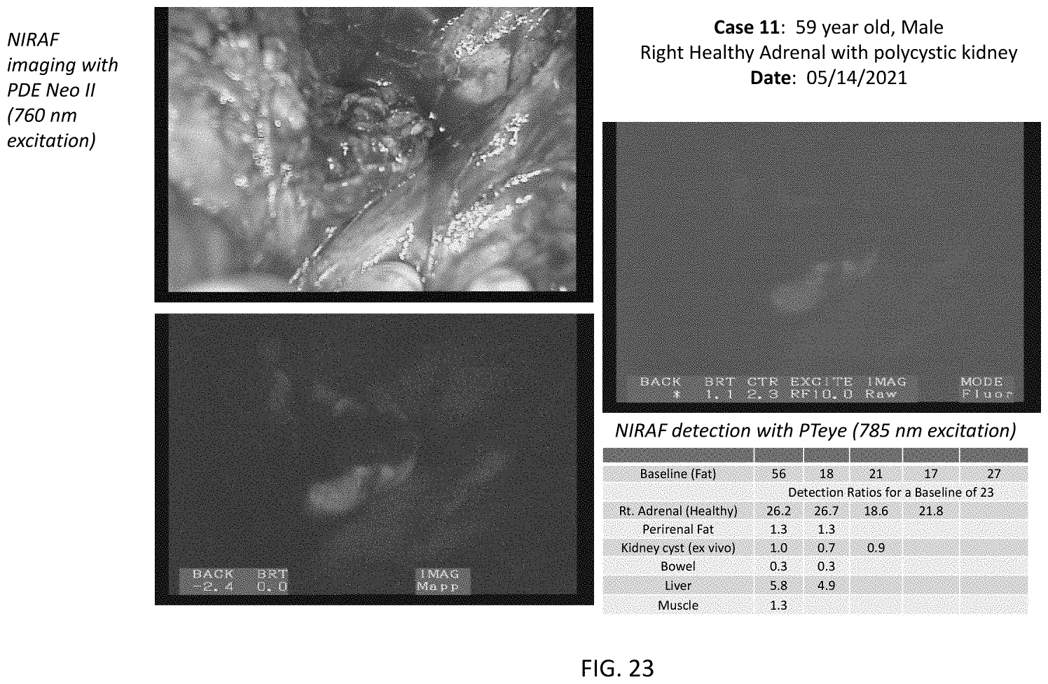

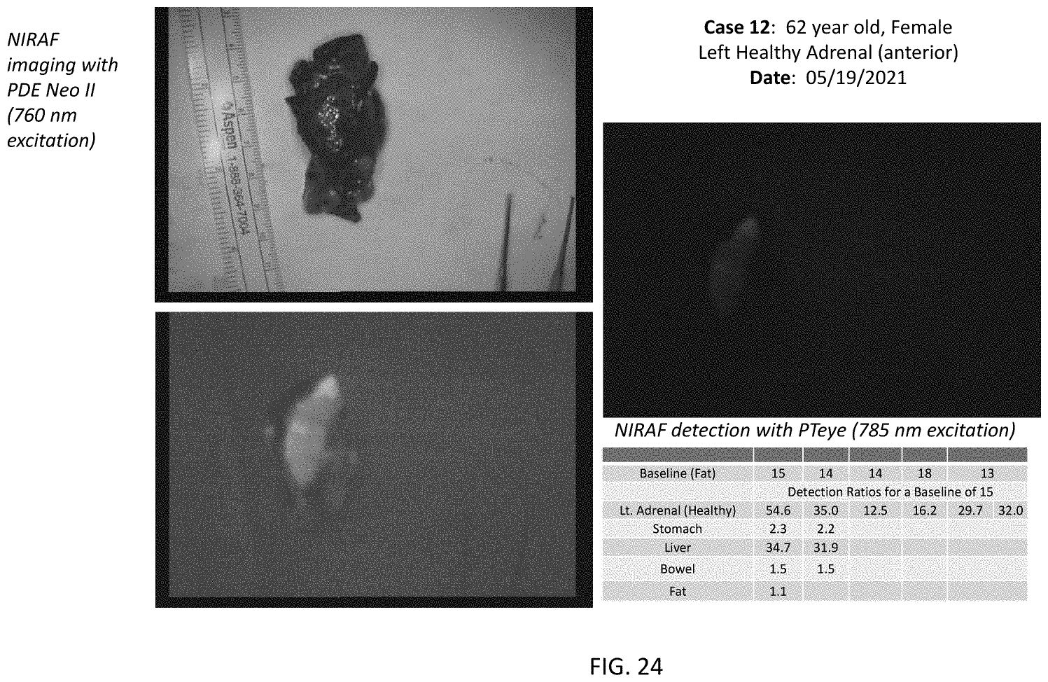

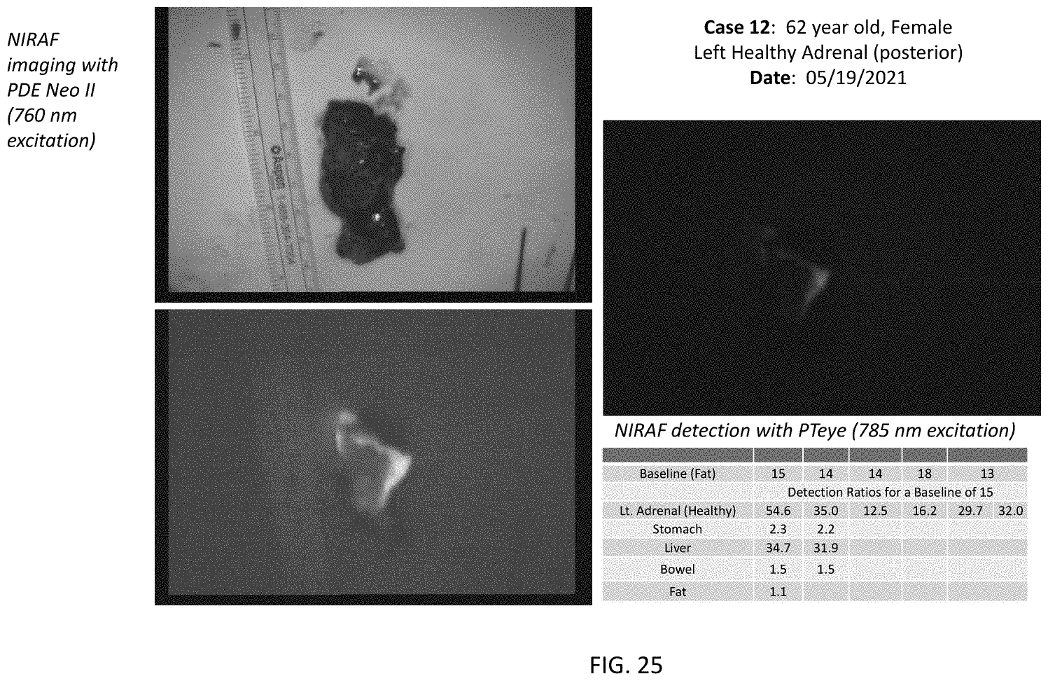

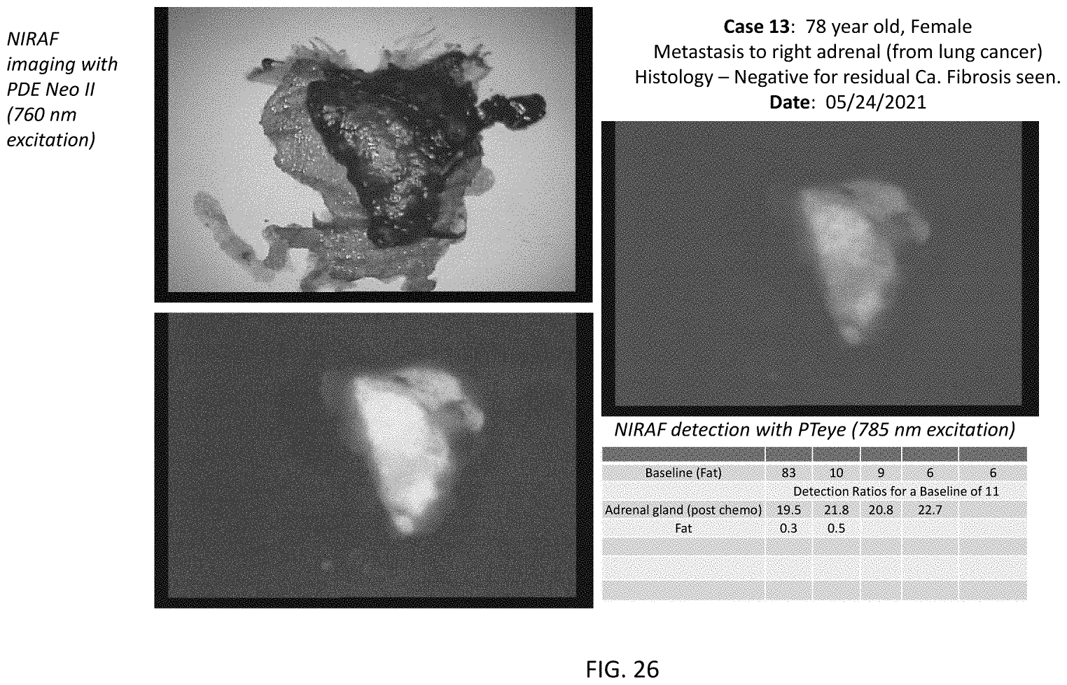

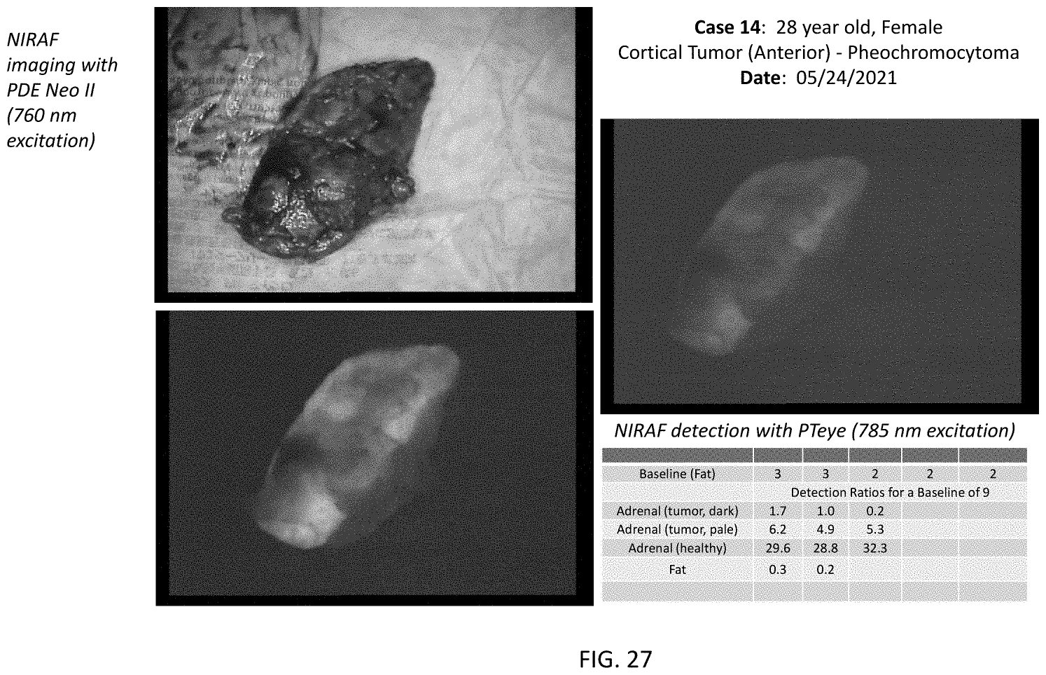

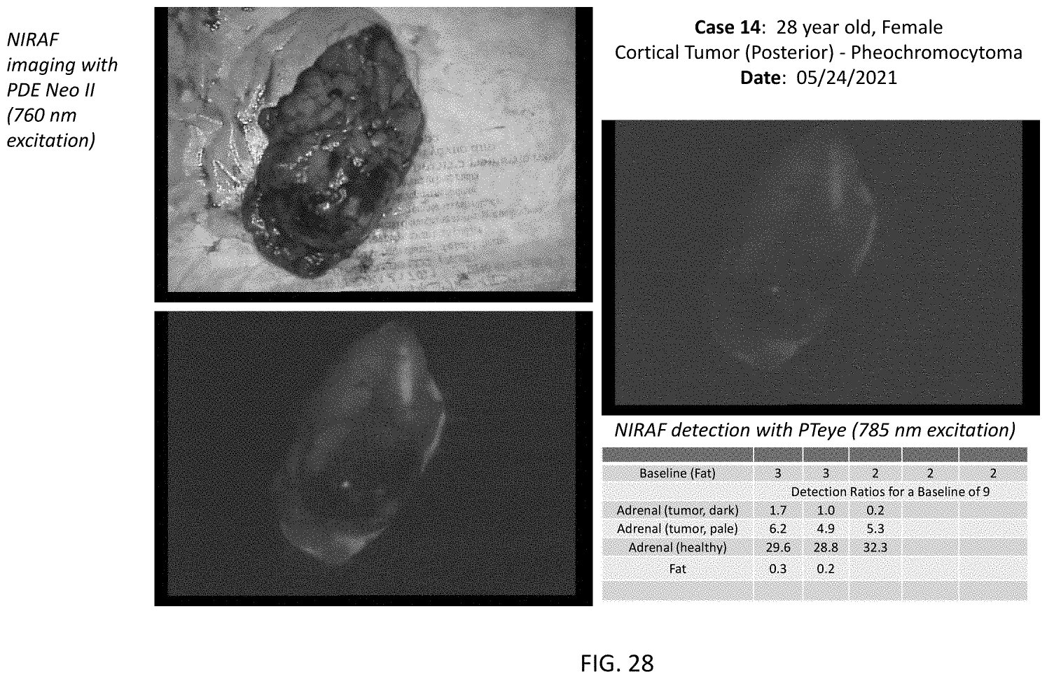

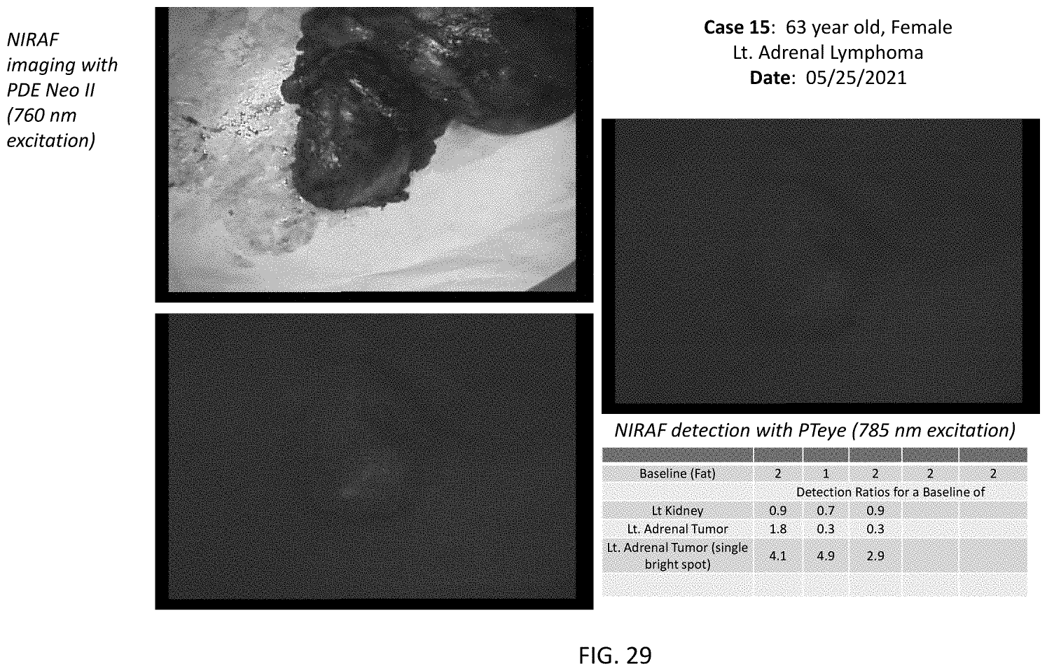

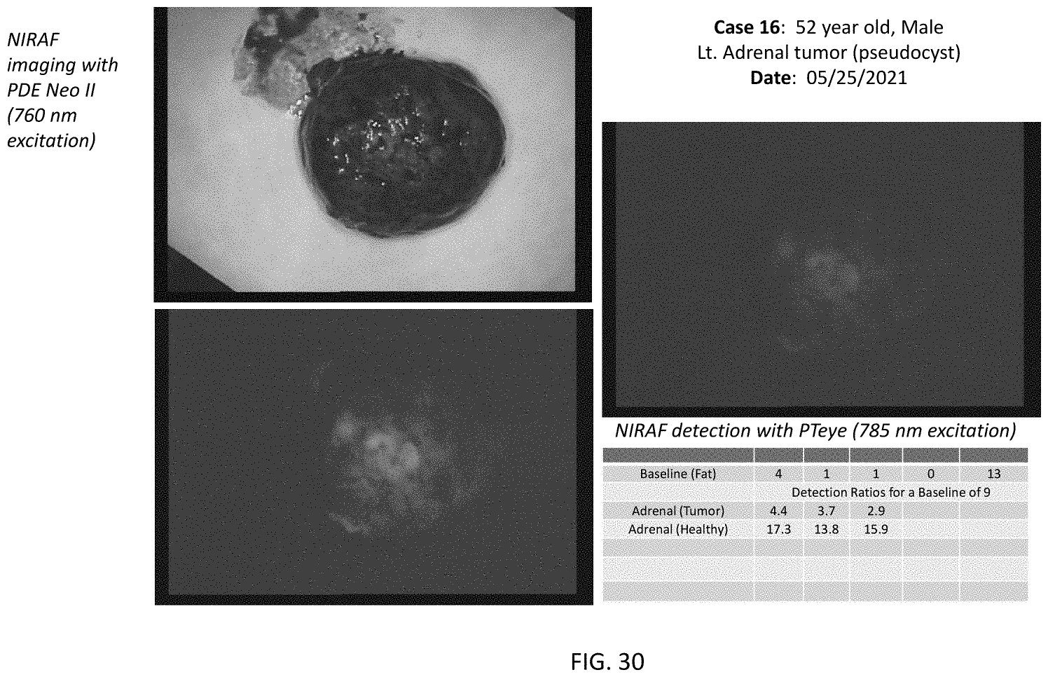

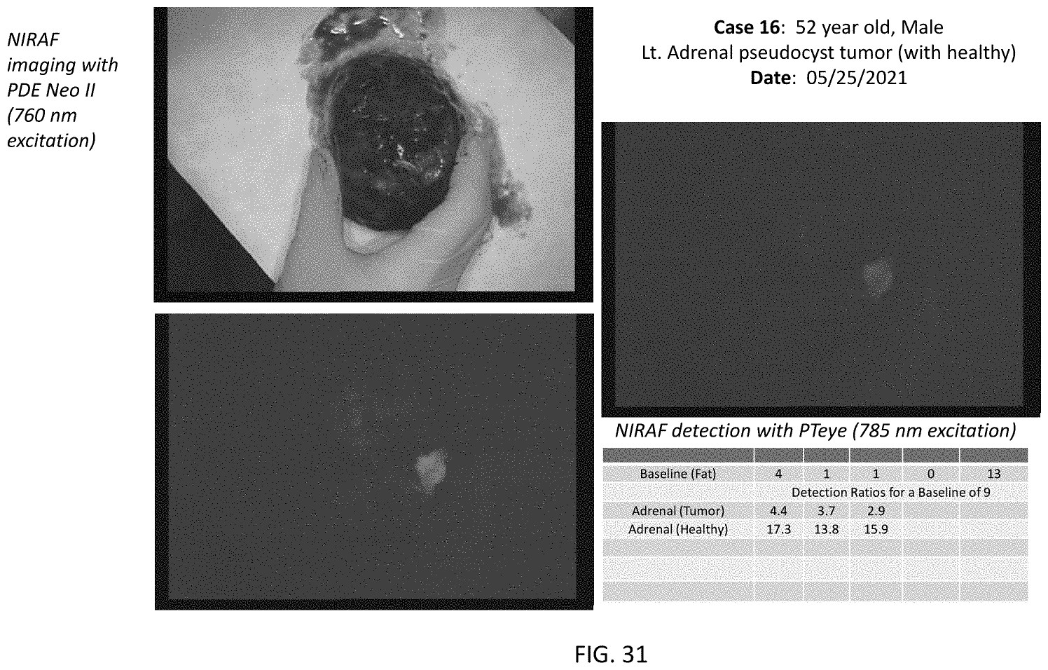

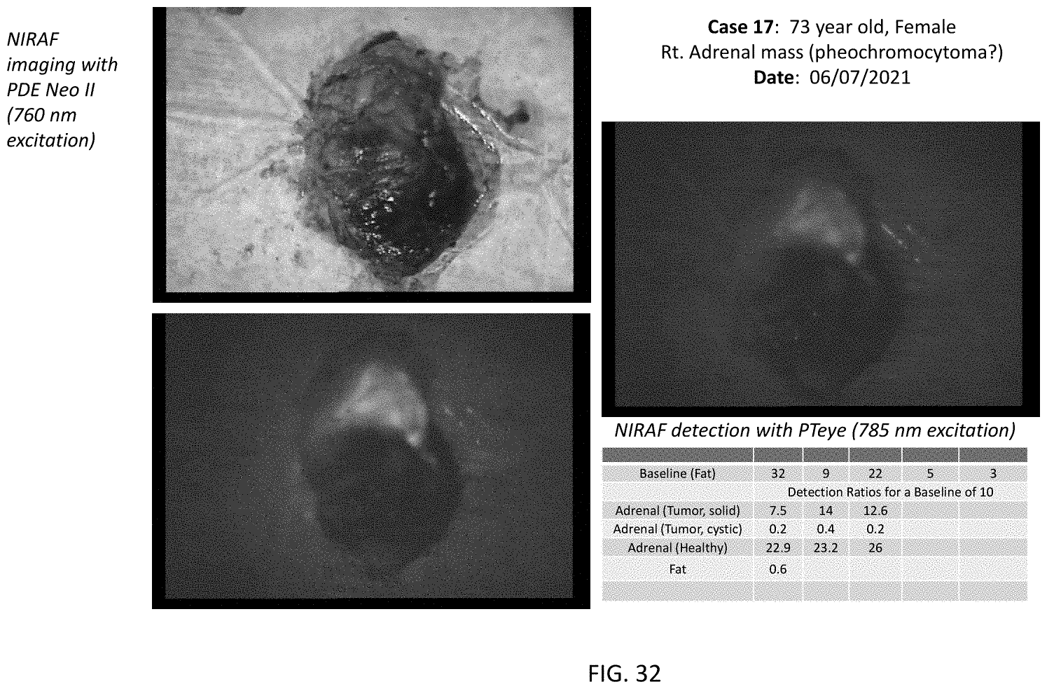

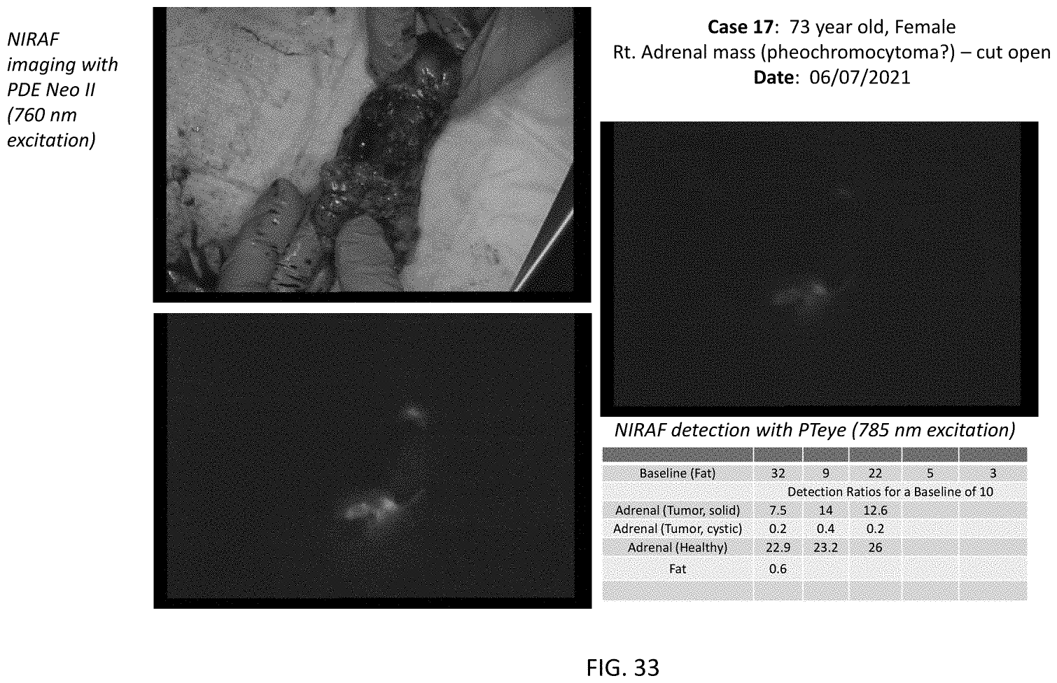

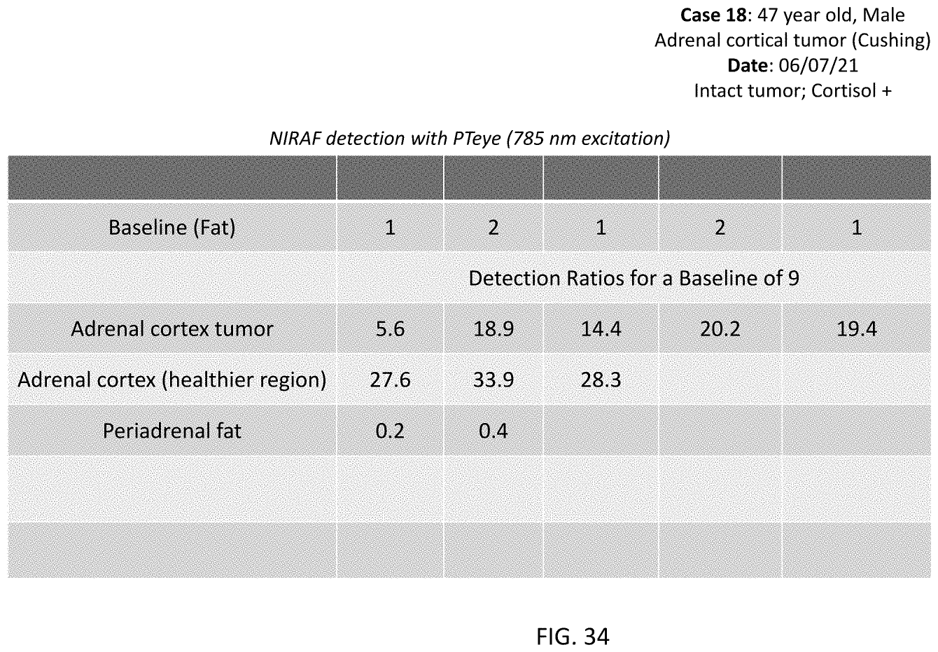

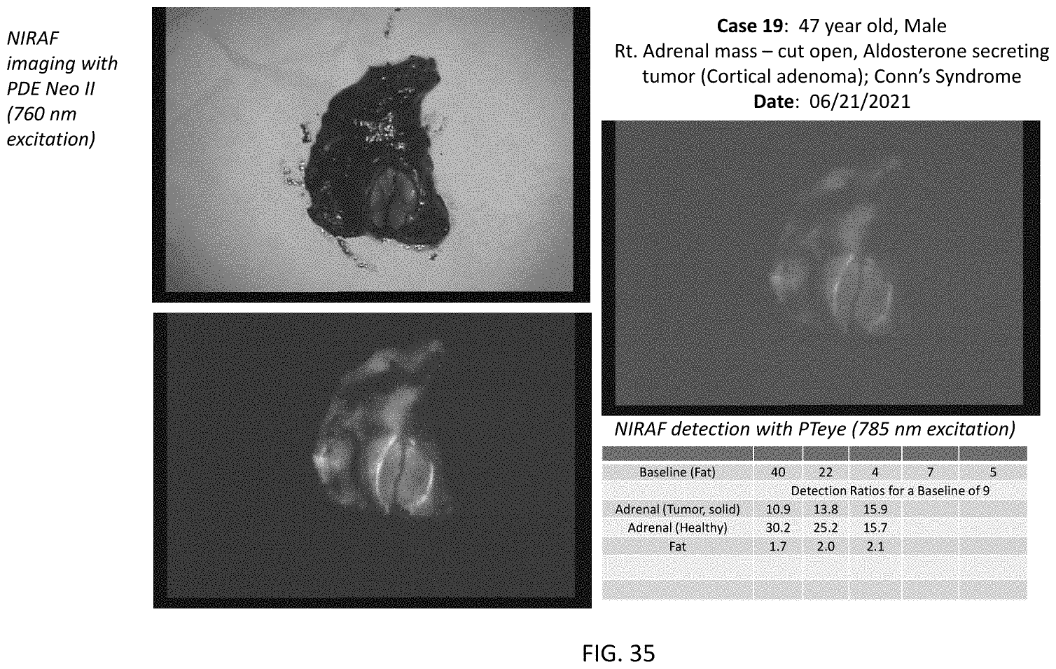

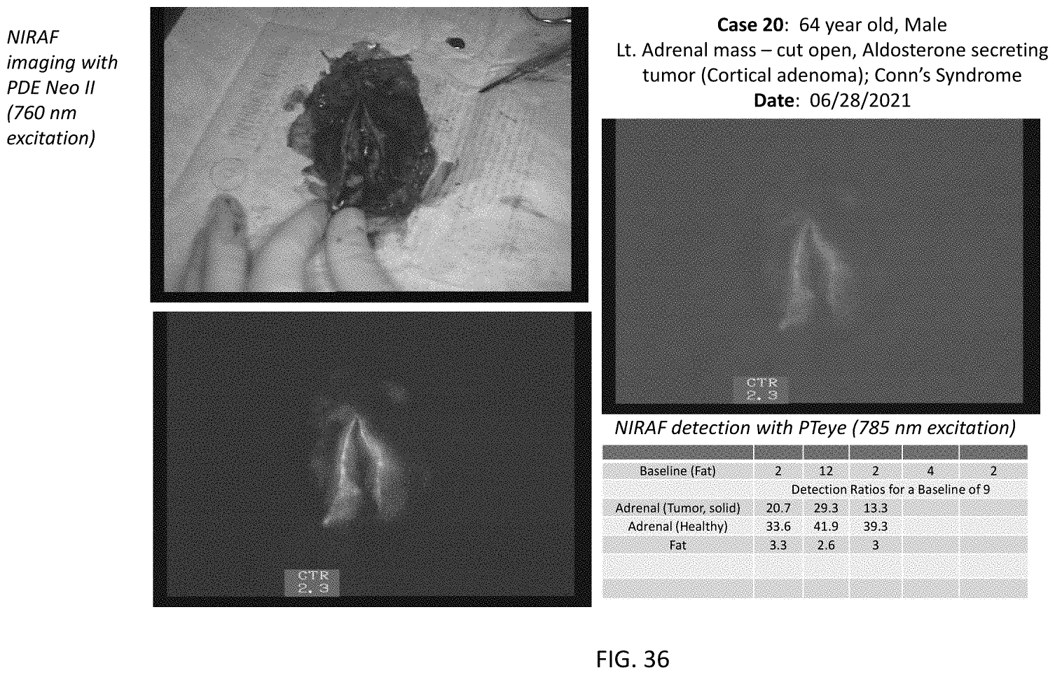

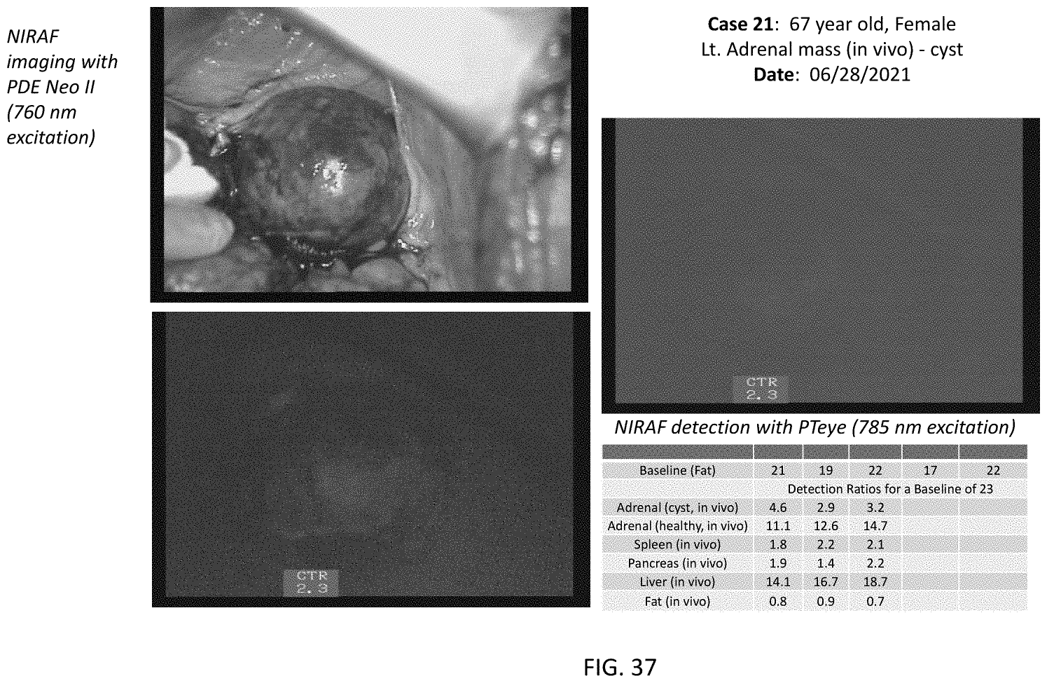

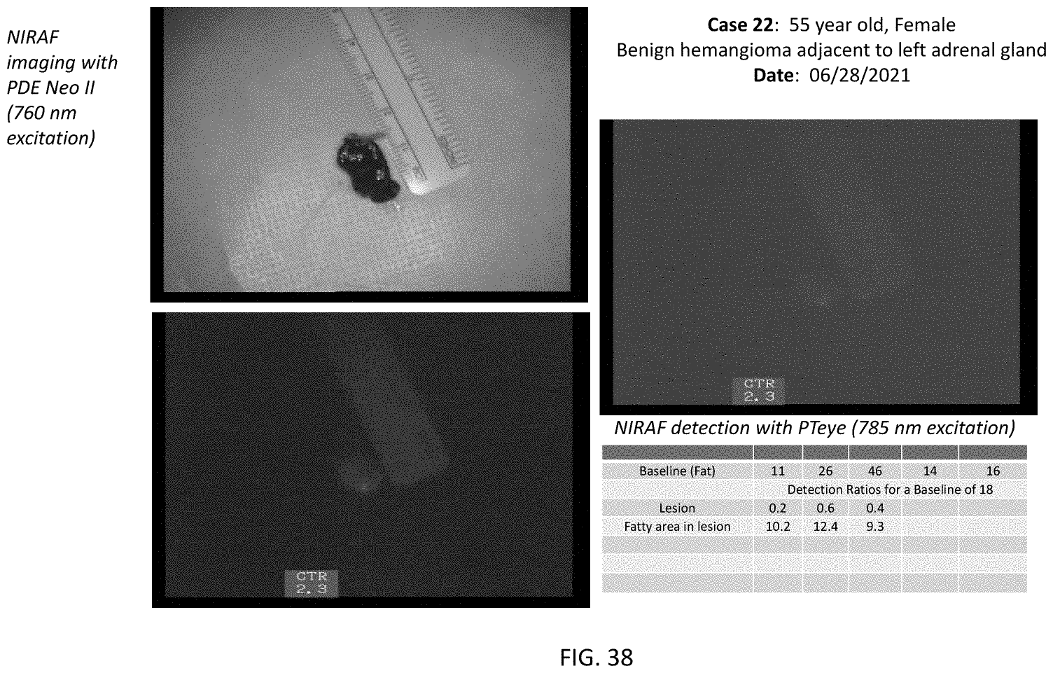

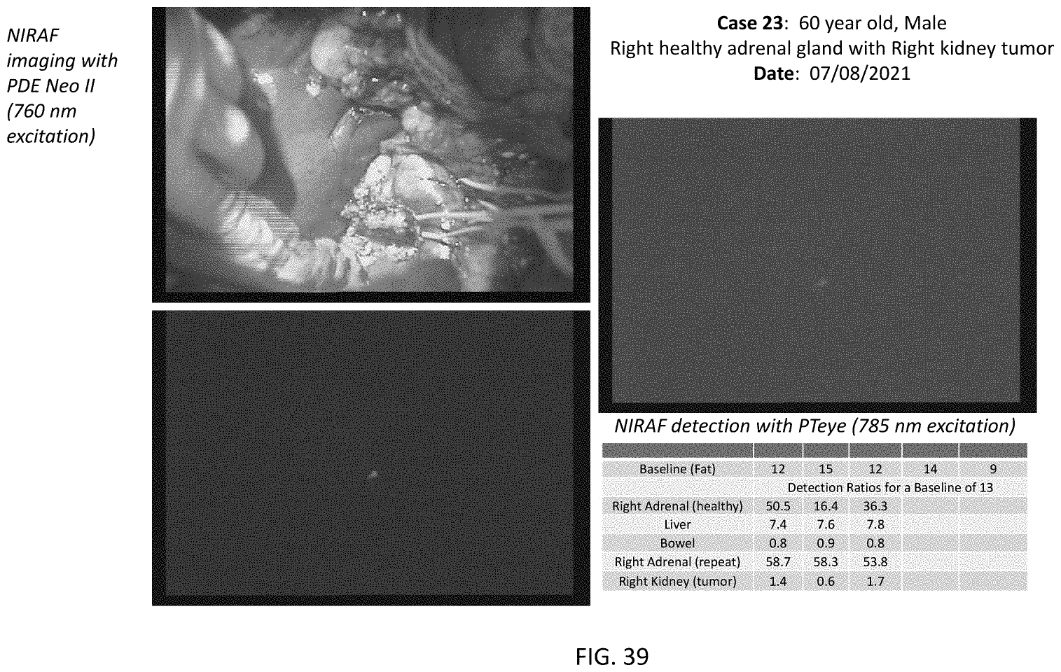

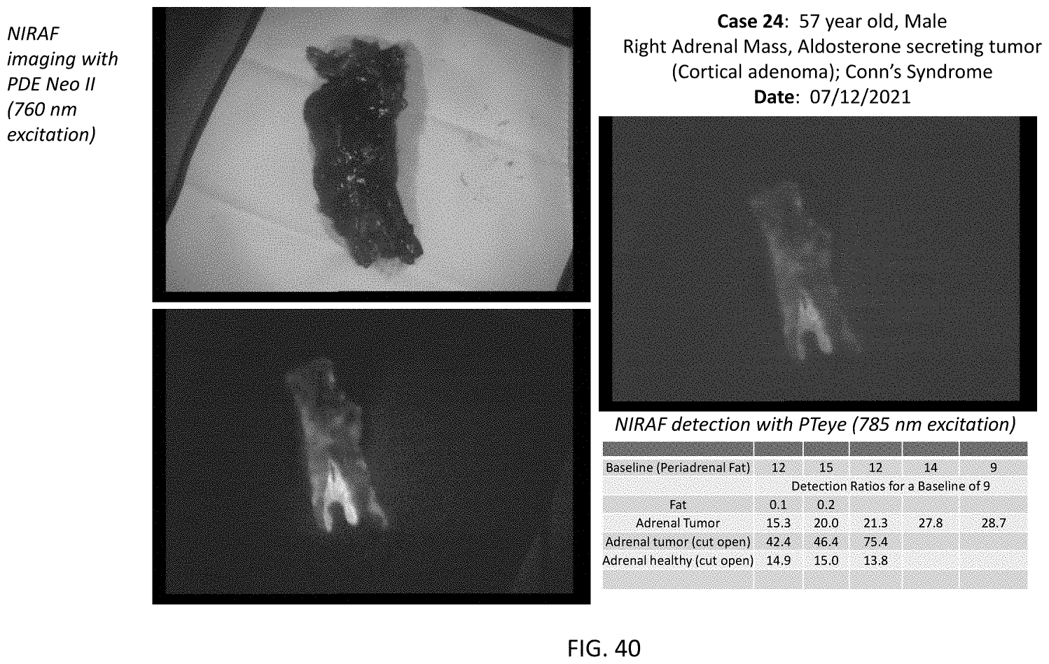

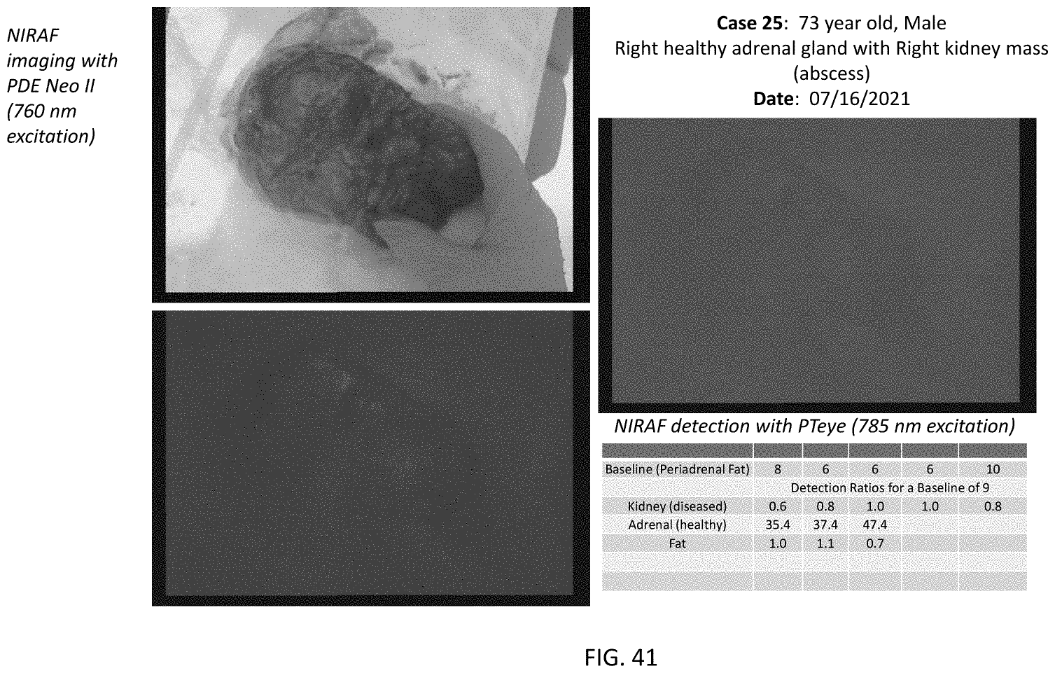

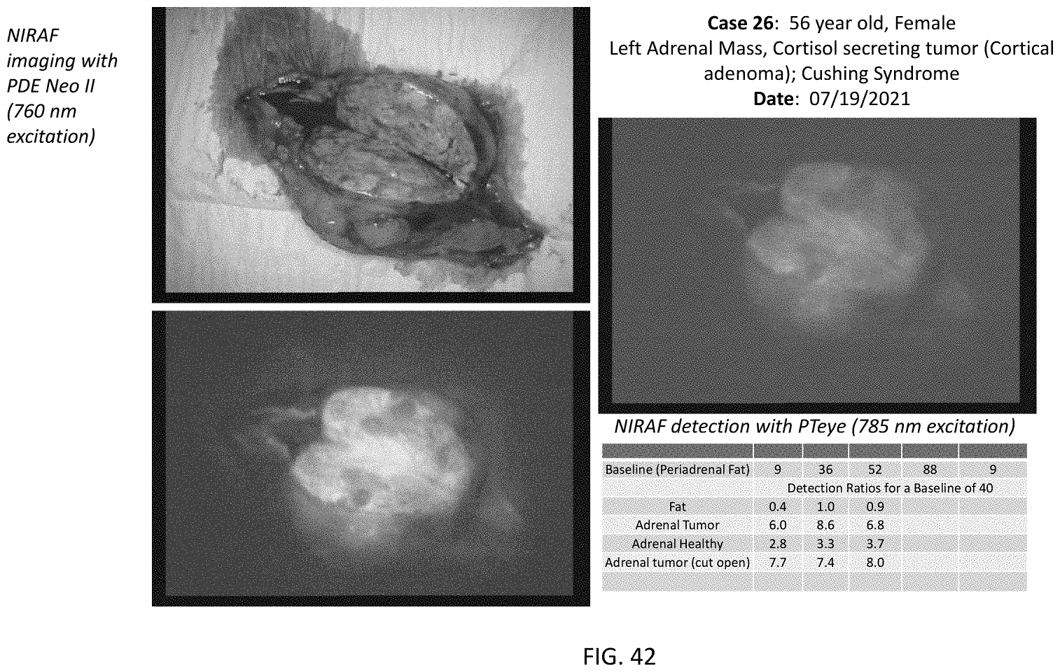

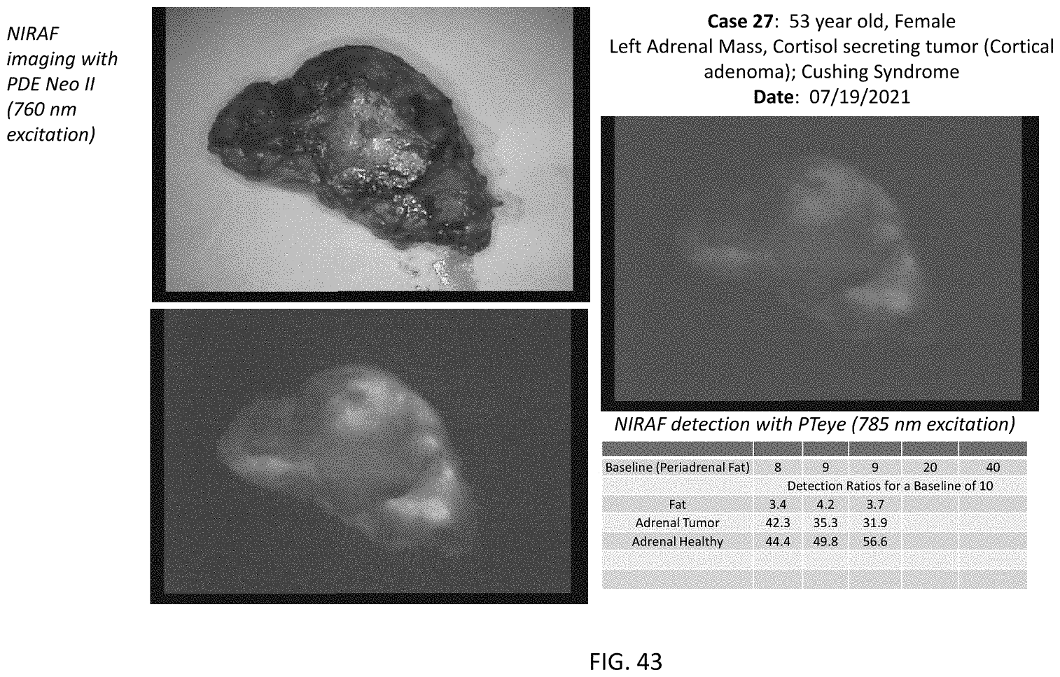

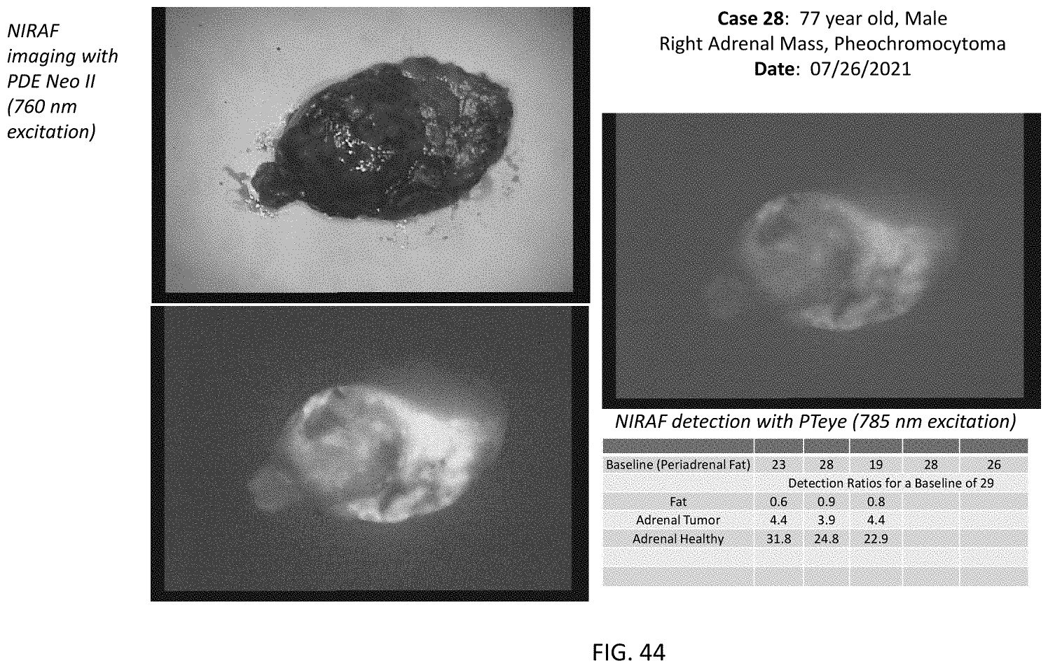

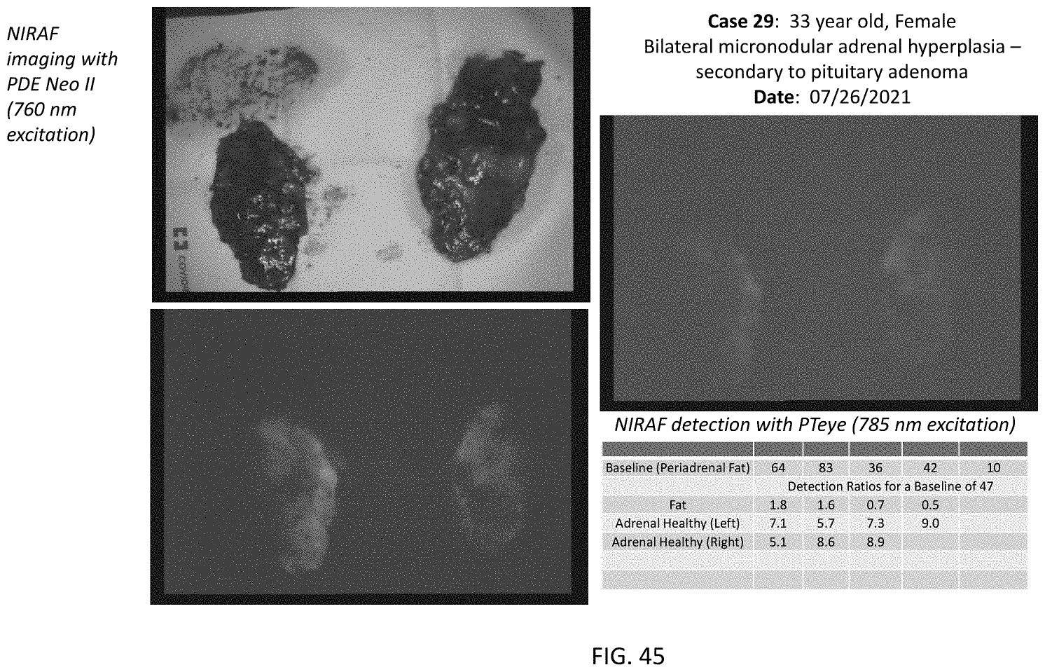

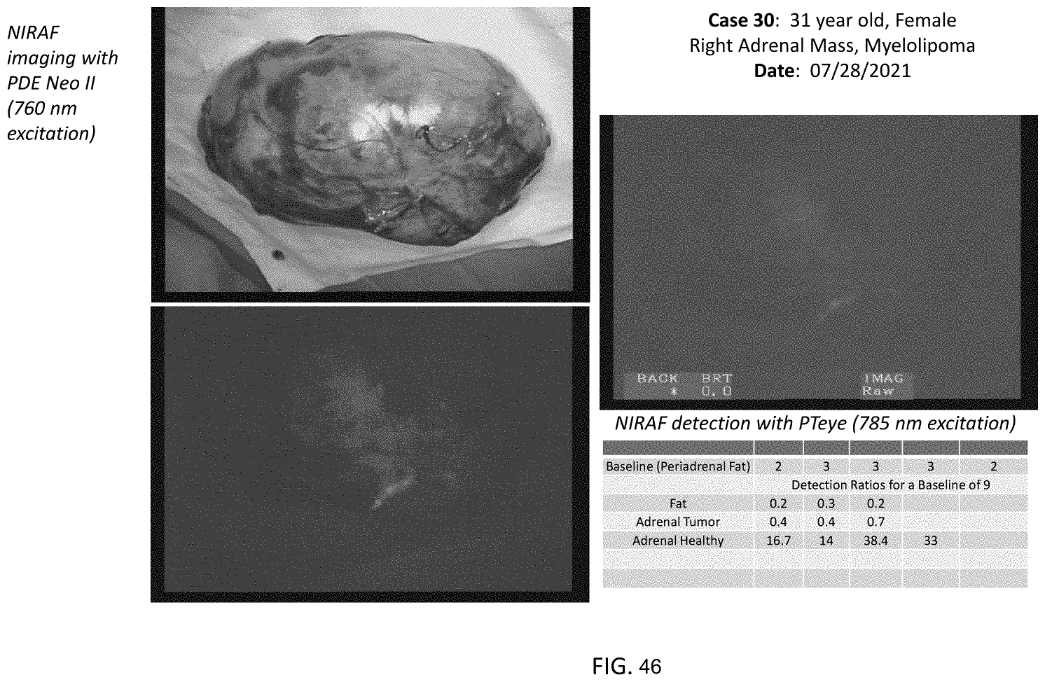

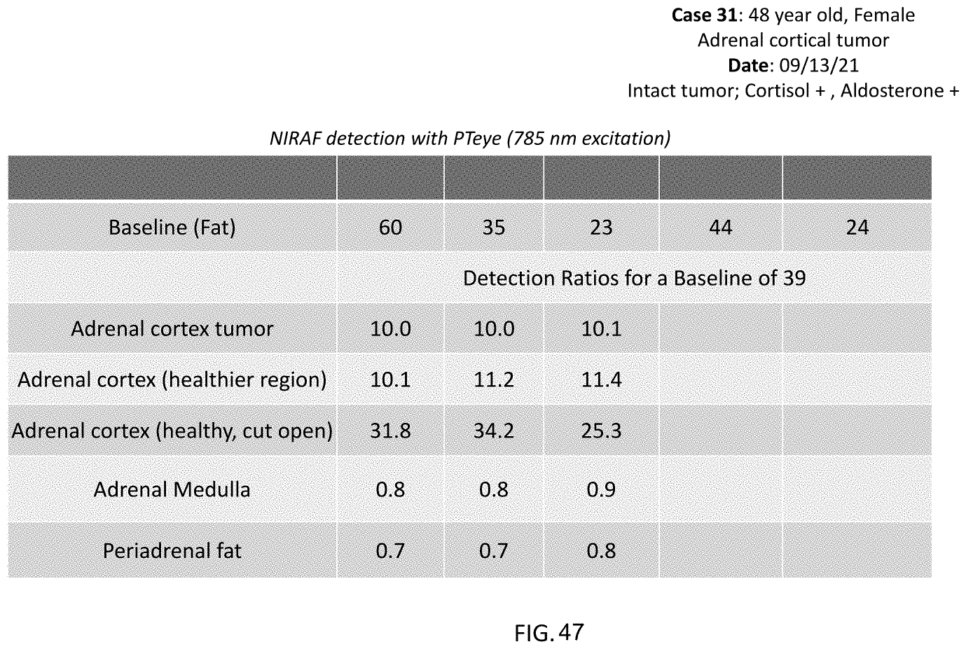

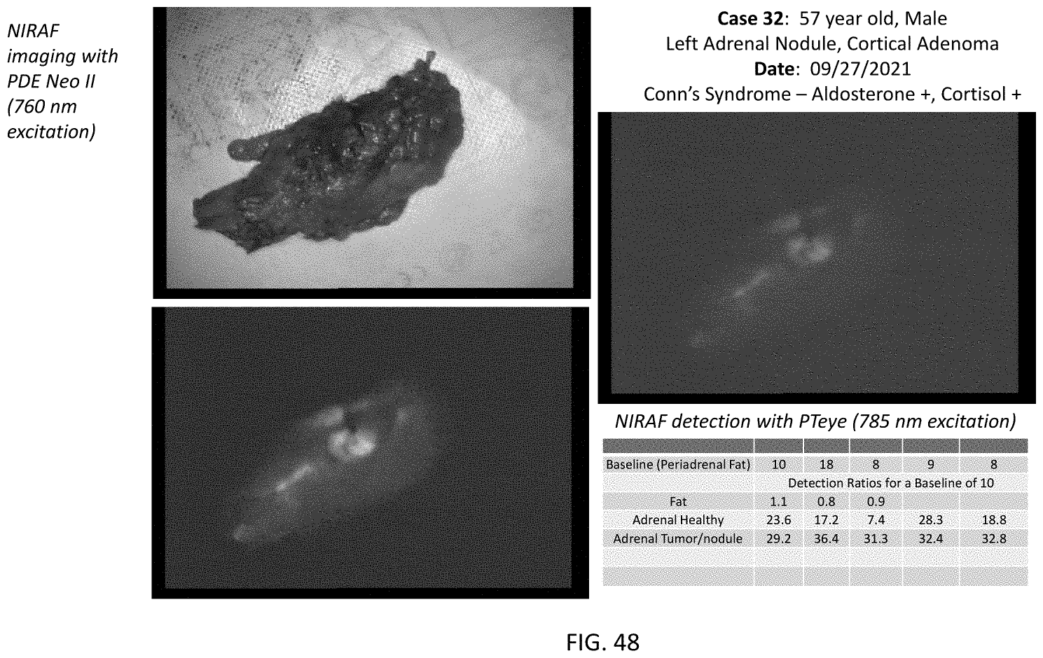

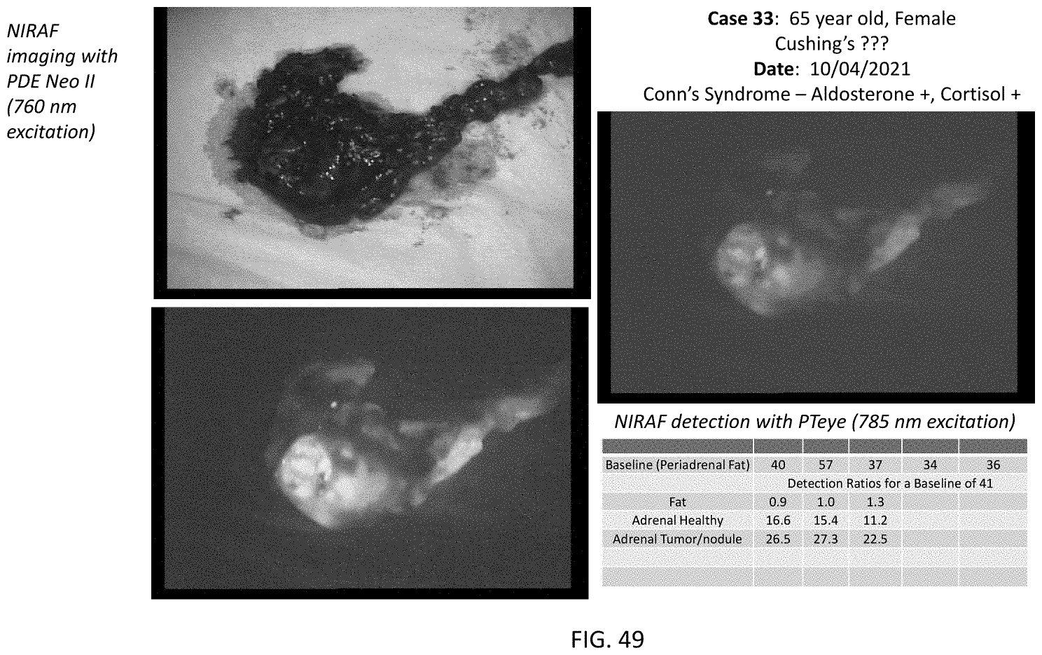

FIGS. 11-49 shows NIRAF acquired from 33 patients accrued with adrenal tumors.

It is concluded, among other things, the NIRAF is significantly elevated in the adrenal gland as compared to the rest of the organs in its surroundings.

NIRAF is the highest in adrenal cortex as compared to adrenal medulla or periadrenal fat.

NIRAF of endocrine tumor in adrenal cortex>>NIRAF of endocrine tumor in adrenal medulla.

NIRAF is present in healthy adrenal as well as diseased adrenal.

NIRAF is low in cysts and non-endocrine tumors of adrenal.

NIRAF can be utilized to localize adrenal glands in the midst of periadrenal fat and bowels, spare healthy adrenal cortex, when resecting tumors arising from adrenal medulla, develop label-free technique, and/or tracking adrenal tumor metastasis to other organs.

Previous literature indicate the need for dyes such as ICG or other targeted agents to localize adrenal glands for resection. Our new findings indicate that it can be done in a label-free manner without ICG or dyes.

### Example 5

### A Novel Label-Free Approach to Enhance Adrenal Gland Visualization Using Near Infrared Autofluorescence Detection During Adrenalectomy

Benign or malignant tumors of the adrenal glands (AGs) are typically managed by adrenalectomy. During adrenalectomy, it is essential to distinguish the AG(s) from retroperitoneal fat and surrounding structures. Traditionally surgeons have either relied on their own subjective visual skills to locate AGs, while ultrasound and exogenous labels have been explored to aid intraoperative AG visualization, all of which have their own limitations. Thereby, we investigated a novel label-free approach using near infrared autofluorescence (NIRAF) detection that could be potentially implemented for enhanced intraoperative AG visualization.

Methods:

Patients undergoing adrenalectomy or nephrectomy were enrolled for this Institutional Review Board-approved study. NIRAF emitted above 800 nm was quantified in-vivo from AGs and surrounding tissues during open adrenalectomy or nephrectomy. Meanwhile for robotic adrenalectomy, NIRAF was similarly measured from excised AGs and other tissue structures ex-vivo. For this study, NIRAF images of tissues were captured using an near infrared (NIR) camera setup, while NIRAF intensities were concurrently recorded using an NIR spectroscopy device. Normalized NIRAF intensities (expressed as mean±standard error) were analyzed and compared, where a p-value<0.05 was considered statistically significant upon using student's t-test.

Results:

Thirty-three patients were enrolled including 15 adrenal cortical tumors, 4 adrenal medullary tumors, 4 adrenal cysts and others-2 adrenal hyperplasia, 1 hemangioma, 1 myelolipoma, 1 lymphoma, 1 secondary metastatic adrenal tumor and 4 healthy AGs. NIRAF intensity measured above 800 nm was significantly elevated for AGs (64.3±7.3) versus retroperitoneal fat (1.9±0.3, p<0.001) and other structures (1.6±0.20, p<0.001). NIRAF images of AGs also revealed a distinct demarcation between adrenal cortex and other periadrenal structures. NIRAF intensity in AGs notably decreased as follows: cortical tumor (88.4±14.3) and healthy cortex (85.1±13.3)>medullary tumor (14.0±3.5)>healthy medulla (1.7±0.8).

Conclusions:

the preliminary findings indicate that NIRAF detection could be a promising technology to enhance AG visualization intraoperatively during adrenalectomy. More importantly, NIRAF detection holds potential for effective delineation between the adrenal cortex and medulla to help cortical-sparing adrenalectomy.

### Example 6

### NIRAF from Patients with Tumors in Small Bowels

This exemplary study was pursued in 6 patients with tumors in small bowels. NIRAF is significantly elevated in the neuroendocrine tumor (n=4 patients) and affected lymph nodes compared to adjacent healthy bowels and abdominal fat. NIRAF was not significantly elevated when the tumor was non-endocrine in nature.

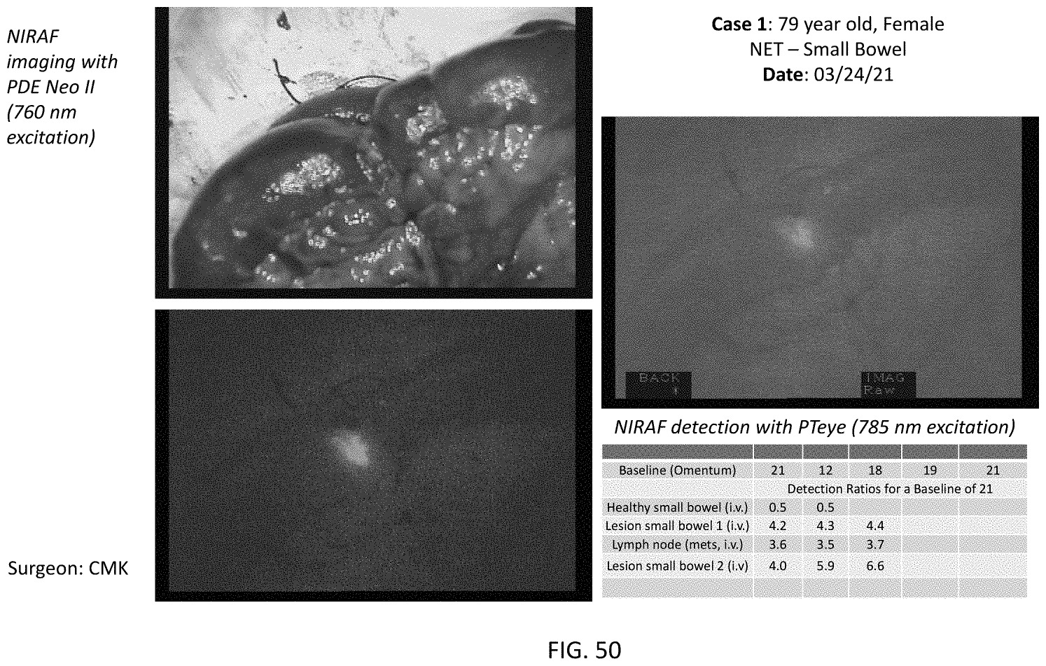

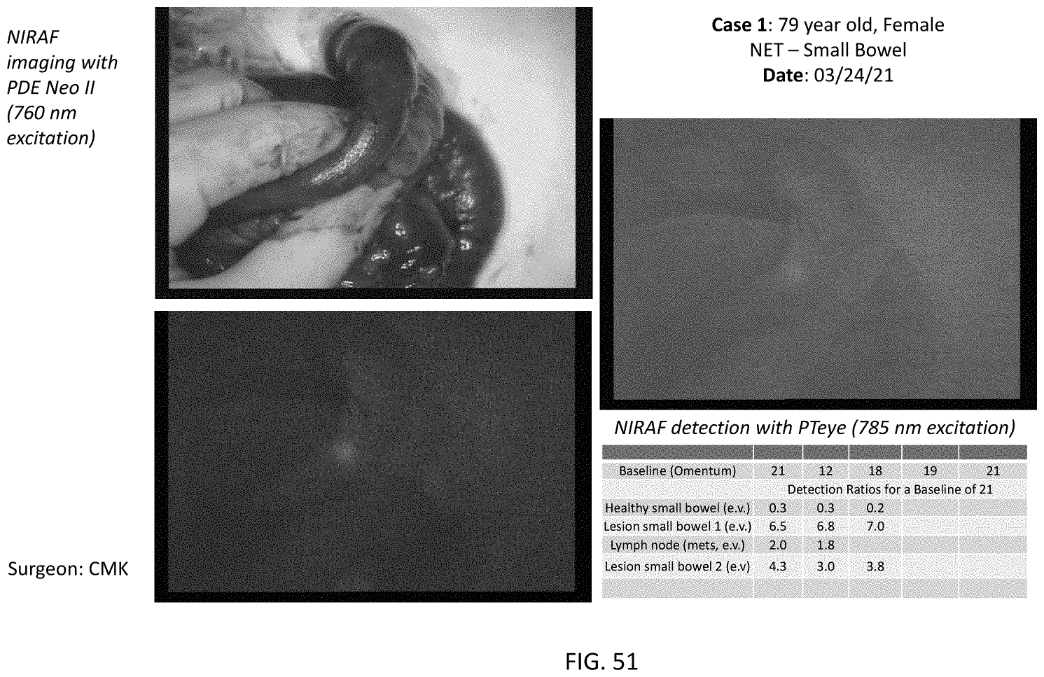

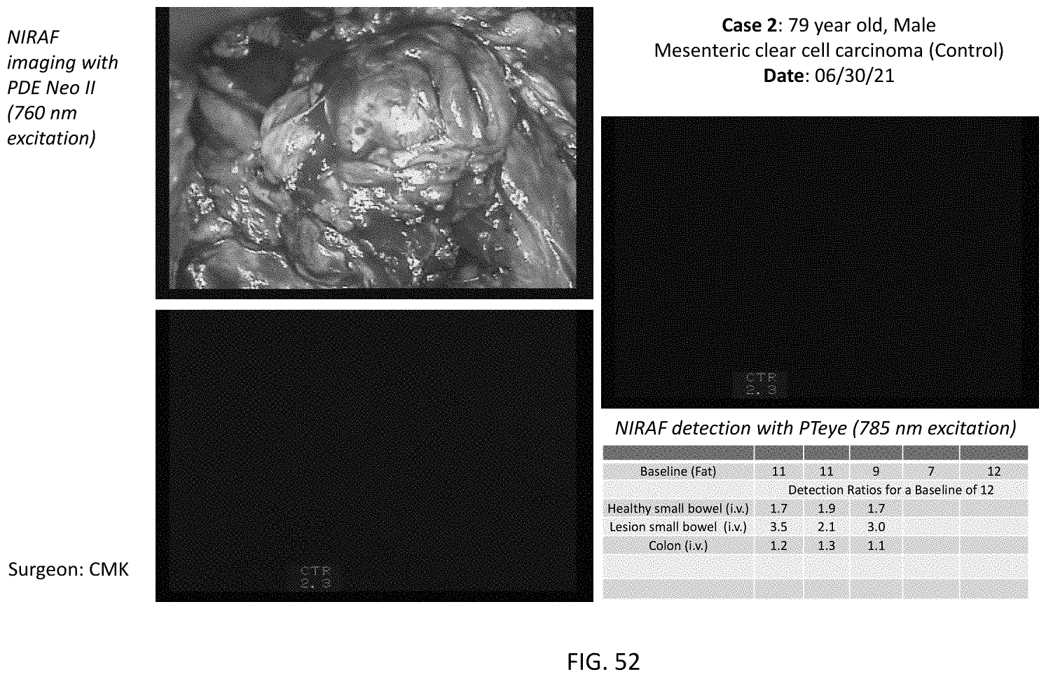

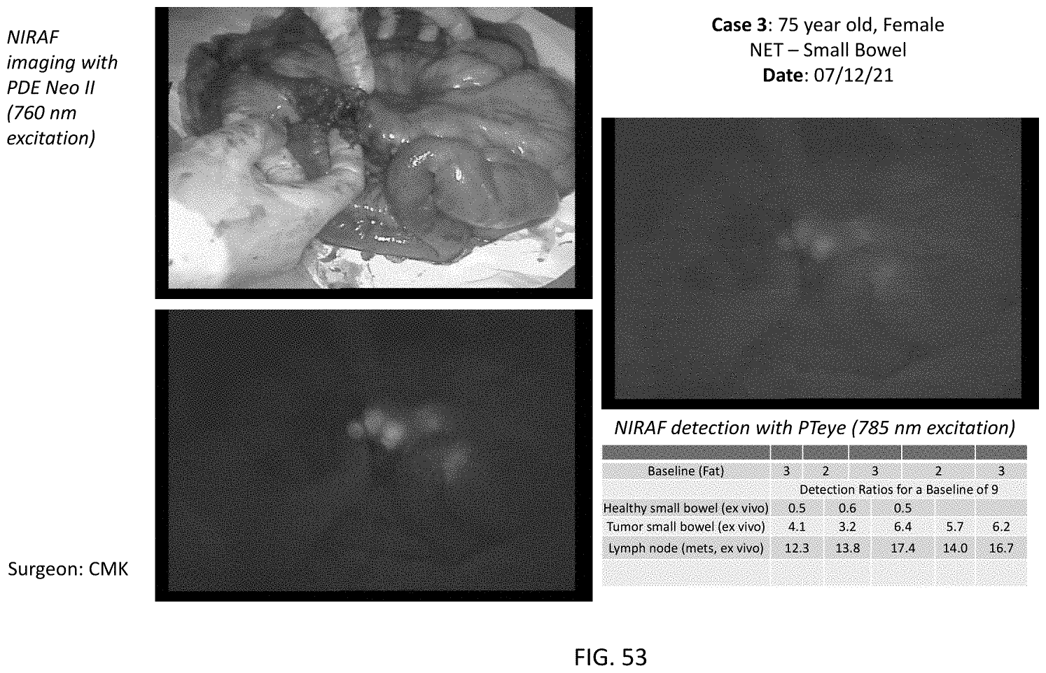

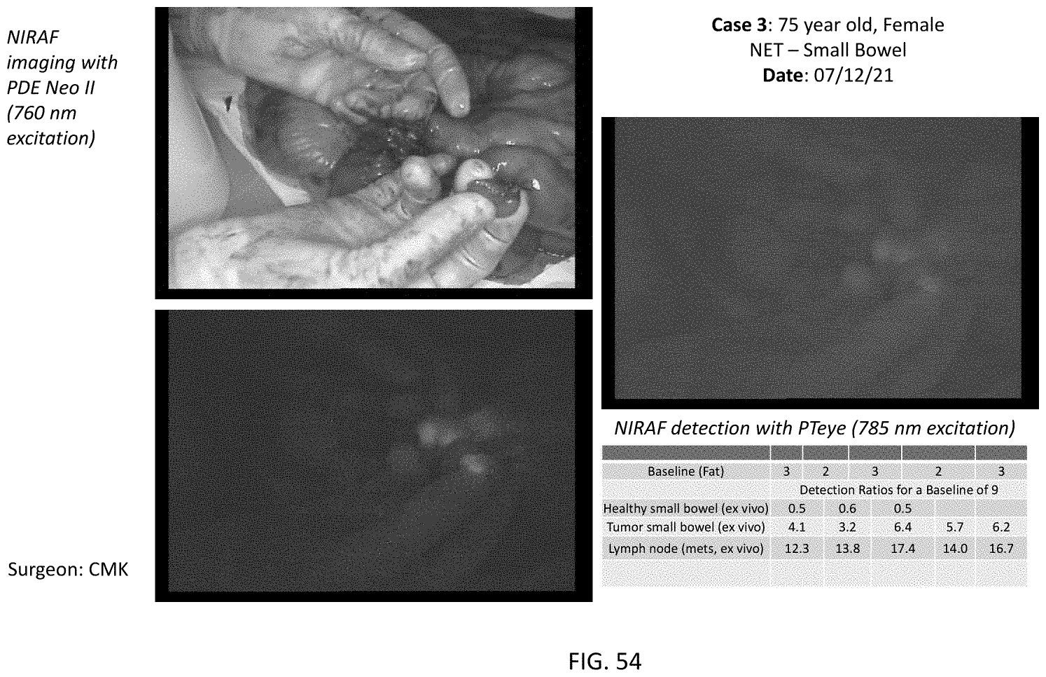

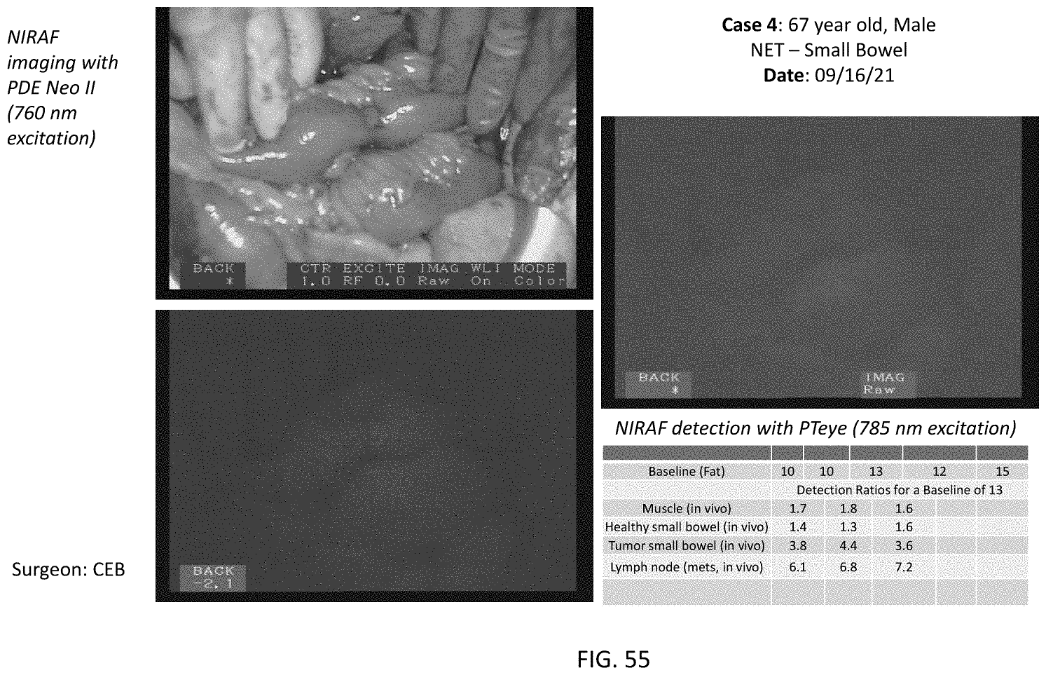

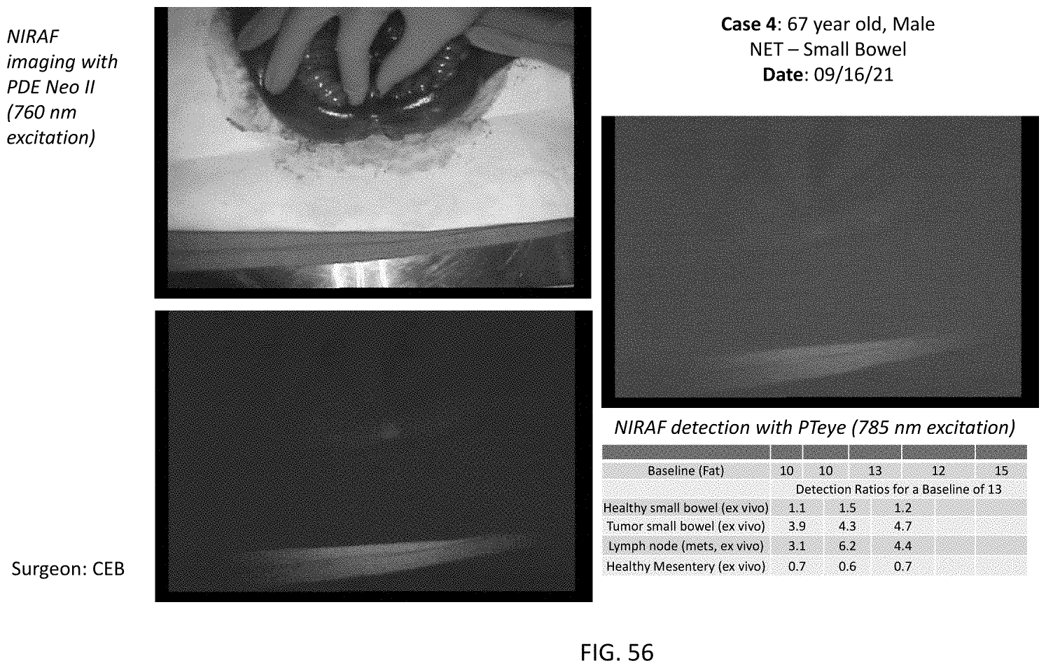

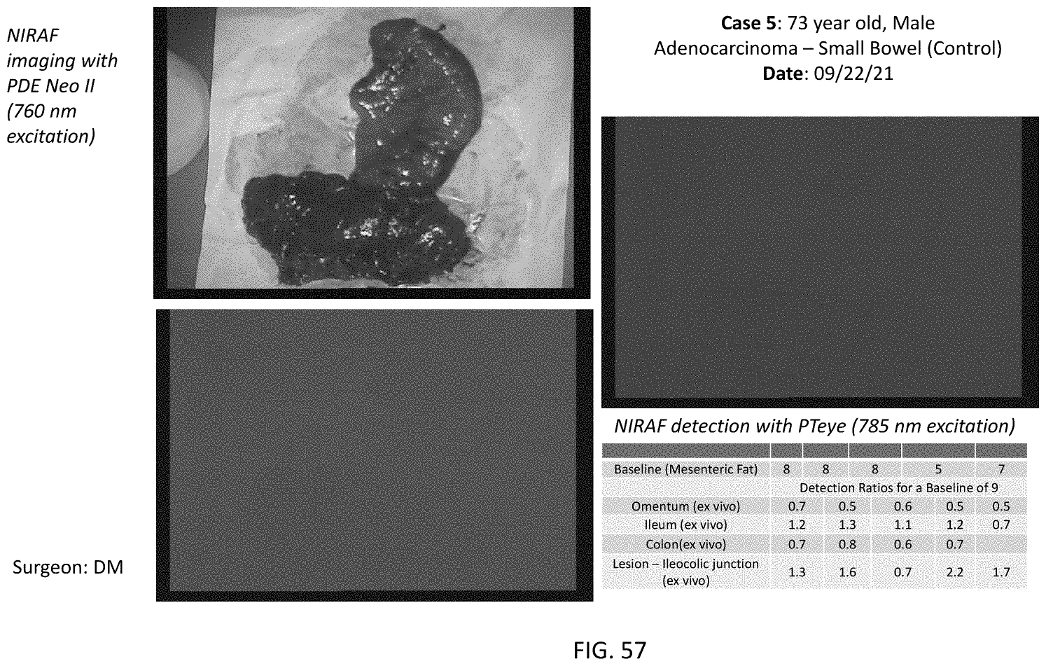

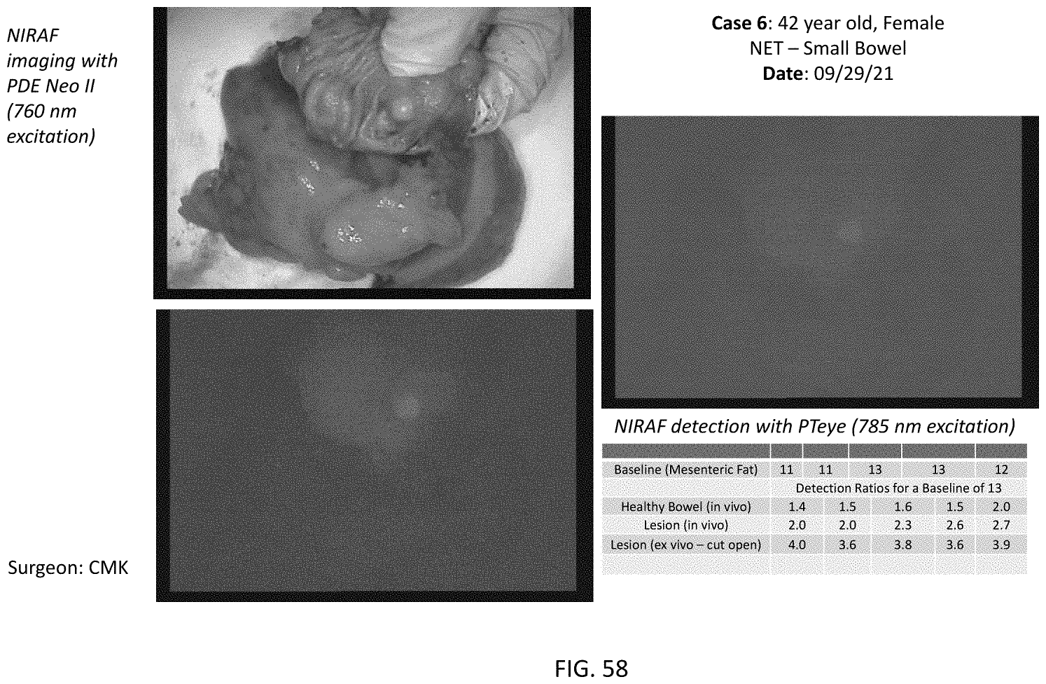

FIGS. 50-58 shows NIRAF acquired from 6 patients with tumors in small bowels.

Among other things, NIRAF can be utilized to localizing and resect neuroendocrine tumor in small bowel, localize and resect lymph nodes with metastasis from small bowel neuroendocrine tumors, and/or localize and track distant metastasis from neuroendocrine tumor of small bowels to vital organs like liver.

Previous literature indicate the need of targeted labels (somatostatin analogues—preoperatively) or dyes (ICG—intraoperatively) for tracking neuroendocrine tumor of small bowels. Our new findings indicate that it can be done in a label-free manner without ICG or dyes.

The foregoing description of the exemplary embodiments of the present invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Heileman, K., Daoud, J., Hasilo, C., Gasparrini, M., Paraskevas, S. and Tabrizian, M., 2015. Microfluidic platform for assessing pancreatic islet functionality through dielectric spectroscopy. Biomicrofluidics, 9(4), p. 044125.
[2]. Wang, Y., Mendoza-Elias, J. E., Lo, J. F., Harvat, T. A., Feng, F., Li, Z., Wang, Q., Nourmohammadzadeh, M., Gutierrez, D., Qi, M. and Eddington, D. T., 2013. Microfluidics for monitoring and imaging pancreatic islet and β-cells for human transplant. In Microfluidic Devices for Biomedical Applications (pp. 557-596e). Woodhead Publishing.
[3]. Newton, A. D., Predina, J. D., Shin, M. H., Frenzel-Sulyok, L. G., Vollmer, C. M., Drebin, J. A., Singhal, S. and Lee IV, M. K., 2019. Intraoperative near-infrared imaging can identify neoplasms and aid in real-time margin assessment during pancreatic resection. Annals of surgery, 270(1), pp. 12-20.
[4]. Paiella, S., De Pastena, M., Landoni, L., Esposito, A., Casetti, L., Miotto, M., Ramera, M., Salvia, R., Secchettin, E., Bonamini, D. and Manzini, G., 2017. Is there a role for near-infrared technology in laparoscopic resection of pancreatic neuroendocrine tumors? Results of the COLPAN “colour-and-resect the pancreas” study. Surgical endoscopy, 31(11), pp. 4478-4484.
[5]. HoáPark, Min, GeorgesáEl Fakhri, and Hak SooáChoi. “Endocrine-specific NIR fluorophores for adrenal gland targeting.” Chemical Communications 52.67 (2016): 10305-10308.
[6]. Yazici, Pinar, Cem Dural, Ryaz Chagpar, Alexis Okoh, Sara Sound, and Eren Berber. “Enhanced Adrenal Gland Visual Contrast by Indocyanine Green Fluorescence.” VideoEndocrinology 2, no. 1.
[7]. Colvin, Jennifer, Nisar Zaidi, and Eren Berber. “The utility of indocyanine green fluorescence imaging during robotic adrenalectomy.” Journal of surgical oncology 114, no. 2 (2016): 153-156.
[8]. Kahramangil, Bora, Emin Kose, and Eren Berber. “Characterization of fluorescence patterns exhibited by different adrenal tumors: determining the indications for indocyanine green use in adrenalectomy.” Surgery 164, no. 5 (2018): 972-977.
[9]. Maxwell, Jessica E., Scott K. Sherman, Yusuf Menda, Donghong Wang, Thomas M. O'Dorisio, and James R. Howe. “Limitations of somatostatin scintigraphy in primary small bowel neuroendocrine tumors.” Journal of surgical research 190, no. 2 (2014): 548-553.
[10]. Rafael, Maria Ana, Ricardo Rocha, Ana Maria Oliveira, Catarina Graca Rodrigues, Carla Carneiro, and Vitor Nunes. “Surgical Resection of Multiple Small Bowel Neuroendocrine Tumours Using Intraoperative Fluorescence Angiography with Indocyanine Green Dye.” Journal of gastrointestinal cancer (2020): 1-4.

What is claimed is:

1. A system for providing anatomical guidance in a surgery, comprising:
a light source configured to emit light having a wavelength in a near-infrared (NIR) range for illuminating tissues of a target region;
means for obtaining autofluorescence signals emitted from the illuminated tissues in response to the illumination; and
a controller configured to process the obtained autofluorescence signals to identify target tissues.

2. The system of claim 1, wherein the light source comprises a laser or an LED configured to emit the light having the wavelength in the NIR range of about 600-1500 nm.

3. The system of claim 1, wherein the means for obtaining the autofluorescence signals comprises:
a light delivery device optically coupled with the light source and configured to deliver the light from the light source to the tissues of the target region; and
a detector configured to collect the autofluorescence signals emitted from the illuminated tissues.

4. The system of claim 3, wherein the light delivery device comprises one or more light guides, or one or more optical fibers.

5. The system of claim 3, wherein the light delivery device comprises a wavelength appropriate notch filter and a double concave lens for beam divergence.

6. The system of claim 3, wherein the light delivery device comprises means for focusing from the light source to the tissues of the target region.

7. The system of claim 3, wherein the means for obtaining the autofluorescence signals further comprises:
at least one lens positioned between the tissues and the detector in an optical path along which the autofluorescence signals emitted from the illuminated tissues travel, and utilized to receive the autofluorescence signals emitted from the tissues and focus them on a desired spot on the detector.

8. The system of claim 7, wherein the means for obtaining the autofluorescence signals further comprises:
at least one filter positioned between the tissues and the at least one lens in the optical path, and utilized to block unwanted wavelengths, ambient light and/or reflected light from the illuminated tissues to ensure the autofluorescence signals to be detected.

9. The system of claim 3, wherein the detector comprises at least one camera, and/or a spectrometer.

10. The system of claim 9, wherein the at least one camera comprises at least one charge-coupled device (CCD) camera, at least one complementary metal oxide semiconductor (CMOS) camera, at least one photosensor array, or a combination thereof.

11. The system of claim 1, further comprising a display for displaying images corresponding to the processed autofluorescence signal for surgical guidance.

12. The system of claim 1, wherein the target region comprises an adrenal gland, a pancreas gland, a thyroid gland, a parathyroid gland, small bowel, or any other organs.

13. The system of claim 1, wherein the surgery is an endocrine surgery.

14. The system of claim 1, wherein identifying the target tissues comprises differentiating diseased tissues from healthy tissues within one organ, or differentiating one organ from other tissues or organs.

15. A system for providing anatomical guidance in surgery, comprising:
a light source configured to emit light having a wavelength in a near-infrared (NIR) range;
an optical means configured to deliver the emitted light to tissues of a target region to illuminate the tissues, and to collect autofluorescence signals emitted from the tissues in response to the illumination;
a detector configured to obtain the collected autofluorescence signals; and
a controller in communication with the detector and configured to process the obtained autofluorescence signals to identify target tissues.

16. The system of claim 15, wherein the optical means comprises an excitation fiber configured to deliver the light emitted from the light source to the tissues, and a plurality of collection fibers aligned circularly around the excitation fiber configured to collect the autofluorescence signals emitted from the tissues in response to the illumination.

17. The system of claim 16, wherein the optical means further comprises an inline filter for preventing intrinsic autofluorescence from the optical fiber itself.

18. The system of claim 17, wherein the optical means further comprise a long-pass filter placed in a fiber port of the detector to further reduce the amount of stray laser light entering the detector.

19. The system of claim 15, wherein the optical means comprises a delivery means for delivering the light emitted from the light source to the tissues, and a collection means for collecting the autofluorescence signals emitted from the tissues in response to the illumination.

20. The system of claim 15, wherein the detector comprises at least one camera, and/or a spectrometer.

21. A method for providing anatomical guidance in surgery, comprising:
illuminating tissues of a target region with light having a wavelength in a near-infrared (NIR) range;
obtaining autofluorescence signals emitted from the illuminated tissues in response to the illumination; and
processing the obtained autofluorescence signals to identify target tissues.

22. The method of claim 21, wherein said processing step comprises finding maximum autofluorescence intensities and/or emission wavelength peaks from the obtained autofluorescence signals.

23. The method of claim 22, wherein the autofluorescence has a highest intensity in cortex of an adrenal gland as compared to that in medulla of the adrenal gland or the periadrenal fat around the adrenal gland.

24. The method of claim 21, wherein said processing step comprises obtaining autofluorescence patterns from the obtained autofluorescence signals; and correlating between the autofluorescence patterns and characteristics of the tissues of the target region.

25. The method of claim 24, wherein said processing step further comprises, when high correlation between the autofluorescence patterns and the characteristics of the tissues of the target region exists, indicating a surgeon to selectively excise specific tissues of the target regions.
